(19) United States
(12) Patent Application Publication
(10) Pub. No.: US 2024/0230662 A9
(43) Pub. Date: Jul. 11, 2024

(54) CALIBRATION OF ELISA FOR CLINICAL ANTIBODY RESPONSE BY QUANTITATIVE MASS SPECTROMETRY

(71) Applicant: Janssen Vaccines & Prevention B.V., Leiden (NL)

(72) Inventors: Jerald C. SADOFF, Washington, DC (US); Ann-Mieke Yvonne, Wilhelmina VERWILLIGEN, Leiden (NL); Daniel John STIEH, Oegstgeest (NL); Eveline SNEEKES-VRIESE, Nieuw-Vennep (NL); Thomas Lodewijk, Gertrudis, Mathilda VAN DEN KERKOF, Amsterdam (NL)

(21) Appl. No.: 18/547,339

(22) PCT Filed: Feb. 25, 2022
(86) PCT No.: PCT/EP2022/054795

§ 371 Date: Aug. 22, 2023

Related U.S. Application Data
(60) Provisional application No. 63/154,128, filed on Feb. 26, 2021.

(30) Foreign Application Priority Data
Apr. 8, 2021 (EP) 21167342.1

(51) Int. Cl. G01N 33/68

(52) U.S. Cl. CPC G01N 33/6848; G01N 33/6854; G01N 2333/135; G01N 2333/16; G01N 2333/165

(57) ABSTRACT

Methods of calibrating ELISA read-out for clinical antibody response are described. In particular, the invention relates to methods for calibrating ELISA read-out for the clinical antibody response to vaccines by quantitative mass spectrometry.

### FIELD OF THE INVENTION

The invention relates to methods of calibrating ELISA read-out for clinical antibody response. In particular, the invention relates to methods for calibrating ELISA read-out for the clinical antibody response to vaccines by quantitative mass spectrometry.

### BACKGROUND OF THE INVENTION

Currently the primary immune response to vaccine products during clinical trials is analyzed by immuno-assays such as ELISA that have a read-out with an arbitrary unit, based on comparison to a reference material of virus reactive, serum which is used in the immune-assay (Aydin 2015). Although this type of read-out gives enough information to compare the immune response of different participants and between groups within the same clinical study, it does not give information on the magnitude of the immune response. Because of this, it is impossible to precisely compare the immune response between various vaccine products, across different target antigens, or to compare clinical date with preclinical data when the exact same assay and reference material are not used.

As to this type of ELISA, there is no practical possibility to move away from immuno-assays and instead use quantitative mass spectrometry as a read-out for the antigen specific antibodies in human serum, because quantitative mass spectrometry cannot differentiate the antigen specific antibodies from other antibodies in human serum.

In contrast to therapeutics where the active pharmaceutical agent can be directly measured in circulation, the pharmacodynamic response of a vaccine is based on the immune system′s response to an immunogen. The concentration of a vaccine-specific antibody in polyclonal serum samples, which can bind to a target antigen in many unique ways, cannot be reliably evaluated against a reference standard based on monoclonal antibodies which have uniform binding stoichiometry and affinities. A reference standard based on polyclonal serum derived from a cohort of people naturally infected or by vaccination will contain a range of antibody specificities at variable concentrations and be a suitable reference for evaluating vaccine responses.

It is possible to use quantitative mass spectrometry to calibrate the clinical immune-assays (Pan, Aebersold et al. 2009), thereby making the read-out of the assays to be an absolute amount in the unit of IgG/IgA per ml serum. This would give an actual magnitude of clinical response to a vaccine and would further enable the possibility to compare it to the response to other vaccine products against the same virus. Further, it would enable the possibility to compare clinical data to preclinical data.

However, the use of mass spectrometry to enable an absolute calibration of a vaccine elicited immune response has not been reported. Although there is a considerable amount of prior art references regarding the establishment of absolute readouts of immune responses, it has relied on the establishment of an accepted standard that can be broadly implemented across laboratories, the use of a purified antibody with a known concentration as a reference standard, or immuno-purification of antibodies followed by a direct quantification.

The ELISA is the most preferred detection method to measure immune response in clinical samples due to its extreme high specificity, high throughput, simplicity, regulatory acceptability and is the only practical solution for the purpose of measuring antigen specific antibodies. Thus, there is a need for the development of a new combination of the two above mentioned techniques, ELISA and quantitative mass spectrometry (qMS).

### BRIEF SUMMARY OF THE INVENTION

The invention addresses this need by calibrating ELISA read-out of clinical antibody response by quantitative mass spectrometry, which allows for the conversion of the arbitrary read-out of ELISA to the absolute amount of antigen specific antibodies. The invention combines two well-known techniques, ELISA and quantitative mass spectrometry (qMS), in such a way that the workhorse of clinical analysis, the ELISA, can be used with all the advantages of qMS, namely, the absolute immunoglobulin read-out (Calderon-Celis, Encinar et al. 2018). Therefore, by using qMS to quantify the response of the ELISA reference material, the arbitrary read-out of the ELISA can be converted to an absolute amount of immunoglobulin.

In a general aspect, the application relates to a method of determining an absolute amount of antibodies specific to an antigen in a sample.

In some embodiments, the method comprises:
i. contacting a calibration material containing the antibodies with the antigen to form one or more protein complexes comprising the antigen and the antibodies bound to the antigen;
ii. determining the relative amount of the antibodies in the calibration material by an ELISA detection method;
iii. repeating step i by contacting the calibration material with the antigen to form one or more protein complexes comprising the antigen and the antibodies bound to the antigen;
iv. isolating the one or more protein complexes;
v. digesting the one or more protein complexes to generate a mixture comprising a proteotypic peptide of the antibodies;
vi. subjecting the mixture to quantitative mass spectrometry to measure a mass response of the proteotypic peptide;
vii. determining the absolute amount of the antibodies in the calibration material by comparing the mass response of the proteotypic peptide to a reference curve of the proteotypic peptide;
viii. establishing a conversion factor to correlate the relative amount of the antibodies in the calibration material as determined by the ELISA in step ii to the absolute amount of the antibodies in the calibration material as determined in step vii; and
ix. applying the conversion factor to determine the absolute amount of the antibodies in the sample.

In some embodiments, the method further comprises obtaining a reference curve of the proteotypic peptide before step i, wherein the reference curve relates a mass response of the proteotypic peptide to an absolute amount of the proteotypic peptide.

In some embodiments, in step vii, the absolute amount of the proteotypic peptide is first determined by comparing the mass response of the proteotypic peptide to the reference curve of the proteotypic peptide, and then the absolute amount of the antibodies in the calibration material is determined based on the absolute amount of the proteotypic peptide.

In some embodiments, the method further comprises:
x. contacting the sample with the antigen to form one or more protein complexes comprising the antigen and the antibodies bound to the antigen;
xi. determining the relative amount of the antibodies in the sample by the ELISA detection method; and
xii. determining the absolute amount of the antibodies in the sample by applying the conversion factor established in step viii.

In some embodiments, the sample is a sample obtained from a subject administered with a vaccine comprising the antigen or a related antigen from the same virus, or from a subject who has been in contact with a virus comprising the antigen or an antigenically related virus.

In some embodiments, the vaccine is a preventive vaccine or therapeutic vaccine, preferably a vaccine against a respiratory syncytial virus (RSV), a vaccine against a human immunodeficiency virus (HIV), a vaccine against an influenza virus, a vaccine against an Ebola virus, a vaccine against a hepatitis A, B, C or D virus, or a vaccine against SARS-COV-2.

In certain embodiments, the antigen is an RSV-B antigen, preferably an RSV-B F antigen.

In certain embodiments, the antigen is an HIV antigen, preferably a Mosaic gp140.

In certain embodiments, the antigen is a SARS-COV-2 antigen, preferably a spike antigen (SARS-COV-2 S).

In some embodiments, the antibodies in the sample bind to the same or different epitopes of the antigen.

In some embodiments, the antibodies comprise one or more of immunoglobulin G (IgG), such as IgG1, IgG2, IgG3 and IgG4.

In some embodiments, the sample is a serum, plasma or another biological fluid sample, preferably human serum.

In some embodiments, the calibration material is a serum, plasma or another biological fluid sample, preferably human serum.

In some embodiments, the antibodies in the sample bind to the same or different epitopes of the antigen that was used as the calibration material.

In some embodiments, the step v further comprises one or more steps of denaturing, reducing, alkylating and/or diluting the one or more protein complexes.

In some embodiments, the proteotypic peptide occurs in the antibodies specific to the antigen, preferably in the Fc region of the antibodies, more preferably comprising the amino acid sequence of SEQ ID NO. 1 or SEQ ID NO. 2.

In some embodiments, the method further comprises subjecting the mixture to liquid chromatography (LC) prior to step vi, preferably the LC is high performance liquid chromatogram (HPLC).

In some embodiments, the quantitative mass spectrometry is a tandem mass spectrometry, preferably comprising multiple reaction monitoring (MRM), selected reaction monitoring (SRM) or a parallel reaction monitoring (PRM).

In certain embodiments, the method comprises measuring the mass response of the intact mass of the proteotypic peptide. In further embodiments, the method further comprises comparing the mass response of the intact mass of the proteotypic peptide to the reference curve of the proteotypic peptide

In another aspect, the application relates to a method of determining an absolute amount of antibodies specific to an RSV antigen in a sample from a subject administered with a vaccine comprising the RSV antigen, the method comprising:
i. contacting the sample with the RSV antigen to form one or more protein complexes comprising the RSV antigen and the antibodies bound to the antigen;
ii. isolating the one or more protein complexes;
iii. digesting the one or more protein complexes to generate a first mixture comprising a proteotypic peptide having the amino acid sequence of SEQ ID NO. 1;
iv. subjecting the first mixture to liquid chromatography (LC), preferably high performance liquid chromatogram (HPLC), to generate a second mixture;
v. subjecting the second mixture to a quantitative mass spectrometry to thereby generate a mass spectrum of the proteotypic peptide;
vi. measuring a mass response of the proteotypic peptide from the mass spectrum; and
vii. determining the absolute amount of the antibodies specific to the RSV antigen in the sample by comparing the mass response of the proteotypic peptide to a reference curve of the proteotypic peptide.

In some embodiments, the method further comprises obtaining a reference curve of the proteotypic peptide before step i, wherein the reference curve relates a mass response of the proteotypic peptide to an absolute amount of the proteotypic peptide.

In some embodiments, the quantitative mass spectrometry is a tandem mass spectrometry, preferably comprising multiple reaction monitoring (MRM), selected reaction monitoring (SRM), or a parallel reaction monitoring (PRM).

In some embodiments, the method comprises measuring the mass response of the intact mass of the proteotypic peptide.

In some embodiments, the method further comprises comparing the mass response of the intact mass of the proteotypic peptide to the reference curve of the proteotypic peptide.

In another aspect, the application relates to a method of determining an absolute amount of antibodies specific to an HIV antigen in a sample from a subject administered with a vaccine comprising the HIV antigen, the method comprising:
i. contacting the sample with the HIV antigen to form one or more protein complexes comprising the HIV antigen and the antibodies bound to the antigen;
ii. isolating the one or more protein complexes;
iii. digesting the one or more protein complexes to generate a first mixture comprising a proteotypic peptide having the amino acid sequence of SEQ ID NO. 1 and a proteotypic peptide having the amino acid sequence of SEQ ID NO. 2;
iv. subjecting the first mixture to liquid chromatography (LC), preferably high performance liquid chromatogram (HPLC), to generate a second mixture;
v. subjecting the second mixture to a quantitative mass spectrometry to thereby generate a mass spectrum of each of the proteotypic peptides;
vi. measuring a mass response of each of the proteotypic peptides from the mass spectrum; and
vii. determining the absolute amount of the antibodies specific to the HIV antigen in the sample by comparing the mass response of each of the proteotypic peptides to a reference curve of each of the proteotypic peptides, wherein the reference curve relates a mass spectrometric response of the proteotypic peptide to an absolute amount of the proteotypic peptide.

In some embodiments, the method further comprises obtaining a reference curve of each of the proteotypic peptides before step i.

In some embodiments, the quantitative mass spectrometry is a tandem mass spectrometry, preferably comprising multiple reaction monitoring (MRM), selected reaction monitoring (SRM), or a parallel reaction monitoring (PRM).

In some embodiments, the method comprises measuring the mass response of the intact mass of each of the proteotypic peptides.

In another aspect, the application relates to a method of determining an absolute amount of antibodies specific to a SARS-COV-2 antigen in a sample from a subject administered with a vaccine comprising the SARS-COV-2 antigen, the method comprising:
i. contacting the sample with the SARS-COV-2 antigen to form one or more protein complexes comprising the SARS-COV-2 antigen and the antibodies bound to the antigen;
ii. isolating the one or more protein complexes;
iii. digesting the one or more protein complexes to generate a first mixture comprising a proteotypic peptide having the amino acid sequence of SEQ ID NO. 1 and a proteotypic peptide having the amino acid sequence of SEQ ID NO. 2;
iv. subjecting the first mixture to liquid chromatography (LC), preferably high performance liquid chromatogram (HPLC), to generate a second mixture;
v. subjecting the second mixture to a quantitative mass spectrometry to thereby generate a mass spectrum of each of the proteotypic peptides;
vi. measuring a mass response of each of the proteotypic peptides from the mass spectrum; and
vii. determining the absolute amount of the antibodies specific to the SARS-COV-2 antigen in the sample by comparing the mass response of each of the proteotypic peptides to a reference curve of each of the proteotypic peptides, wherein the reference curve relates a mass spectrometric response of the proteotypic peptide to an absolute amount of the proteotypic peptide.

In some embodiments, the method further comprises obtaining a reference curve of each of the proteotypic peptides before step i.

In some embodiments, the quantitative mass spectrometry is a tandem mass spectrometry, preferably comprising multiple reaction monitoring (MRM), selected reaction monitoring (SRM), or a parallel reaction monitoring (PRM).

In some embodiments, the method comprises measuring the mass response of the intact mass of each of the proteotypic peptides.

Other aspects, features and advantages of the invention will be apparent from the following disclosure, including the detailed description of the invention and its preferred embodiments and the appended claims.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 shows mass response of peptide VVSVLTVLHQDWLNGK (SEQ ID NO. 1) as described in Example 1. The peak area is expressed in relative abundance.

FIG. 2 shows an ELISA reference standard curve for anti-RSV-B antigen antibody positive human serum bound to RSV-B F antigen, obtained by spectrophotometric read-out. Luminescence is expressed in Relative light units (RLU).

FIG. 3 discloses the experimental set up of qMS analysis of RSV-B F antigen positive human serum, which is the ELISA reference material.

FIG. 4 discloses the linear part of the ELISA reference standard curve, generated with anti-RSV-B F antigen antibody positive human serum versus the amount of antigen bound IgG read-out by mass spectrometry.

FIG. 5 shows mass response of peptide VVSVLTVLHQDWLNGK (SEQ ID NO. 1) and VVSVLTVVHQDWLNGK (SEQ ID NO.2) as described in Example 2. The peak area is expressed in relative abundance.

FIG. 6 shows an ELISA reference standard curve for anti-HIV antigen (Mosaic gp140) antibody positive human serum bound to HIV antigen, obtained by spectrophotometric read-out. The read-out is expressed in Optical Density at 450 nm wave length (0D450).

FIG. 7 discloses the linear part of the ELISA reference standard curve, generated with anti-HIV antigen antibody positive human serum versus the amount of antigen bound IgG read-out by mass spectrometry.

FIG. 8 shows mass response of peptide VVSVLTVLHQDWLNGK (SEQ ID NO. 1) and VVSVLTVVHQDWLNGK (SEQ ID NO.2) as described in Example 3. The peak area is expressed in relative abundance.

FIG. 9 shows an ELISA reference standard curve for anti-SARS-COV-2 antigen antibody positive human serum bound to SARS-COV-2 antigen, obtained by spectrophotometric read-out. Luminescence is expressed in Relative light units (RLU).

FIG. 7 discloses the linear part of the ELISA reference standard curve, generated with anti-SARS-COV-2 antigen antibody positive human serum versus the amount of antigen bound IgG read-out by mass spectrometry.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications, and publications cited herein are incorporated by reference as if set forth fully herein.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the application can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise stated, any numerical value, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1 mg/mL to 10 mg/mL includes 0.9 mg/mL to 11 mg/mL. As used herein, the use of a numerical range expressly includes all possible subranges, all individual numerical values within that range, including integers within such ranges and fractions of the values unless the context clearly indicates otherwise.

As used herein, “subject” means any animal, preferably a mammal, most preferably a human, to who will be or has been treated by a method according to an embodiment of the application. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, humans, etc., more preferably a human.

In an attempt to help the reader of the application, the description has been separated in various paragraphs or sections and/or in various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples.

The term “biological sample” as used herein encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. The antibody can be monomeric or multimeric and can be derived from any species and can be IgM, IgG (e.g. IgG1, IgG2, IgG3, or IgG4), IgD, IgA, or IgE, for example.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdab) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure.

The term “monoclonal antibody (mAb)” as used herein refers to a preparation of antibodies of single molecular composition.

The term “polyclonal antibody” as used herein refers to a composition of different antibody molecules which is capable of binding to or reacting with several different specific antigenic determinants on the same or on different antigens. The variability in antigen specificity of a polyclonal antibody is located in the variable regions of the individual antibodies constituting the polyclonal antibody, in particular in the complementarity determining regions CDR1, CDR2 and CDR3.

The term “antigen specific antibody” refers to binding between an antigen and an antibody, characterized by the ability of the antigen to associate with the antibody even in the presence of many other diverse antibodies, i.e., to show preferential binding in a heterogeneous mixture of antibodies. For example, an antigen specific antibody refers to an antibody or an antigen binding fragment thereof that binds to the antigen with a KD of 1×10^{31 7} M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for a binding agent can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system. The smaller the value of the KD of an antibody, the higher affinity that the antibody binds to a target antigen.

The term “absolute amount” of antibodies refers to the amount in the units of weight, moles, weight concentration, or molar concentration, preferably weight concentration or molar concentration.

The term “vaccine” refers to a biological preparation that provides active acquired immunity to a particular infectious disease. A vaccine typically contains an agent that resembles a disease-causing microorganism including virus. For example, a human immunodeficiency virus (HIV) vaccine contains or encodes for one or more portions/fragments of HIV virus, and the portion/fragment of HIV virus is also called HIV antigen. Vaccines can be prophylactic (to prevent or ameliorate the effects of a future infection by a natural or “wild” pathogen), or therapeutic (e.g., vaccines against cancer, which are being investigated).

### ELISA for the Determination of Immune Response

ELISA is the most preferred detection method to measure immune response in human serum samples due to its extreme high specificity, high throughput and simplicity, and its ability to measure antigen specific antibodies. Indirect ELISA, detecting serum antibodies with the help of a species-specific secondary antibody, is commonly used for this purpose. The first step in an indirect ELISA is the immobilization of the target antigen in wells of a microtiter plate. Antigen can unspecifically (adsorption) or specifically (via e.g. streptavidin) be adsorbed to the wells of the microtiter plate. The detection of antigen specific antibodies is a two-step process: first, serum or plasma antibodies or purified and unlabeled primary antibodies bind to the specific antigen; second, a secondary detection antibody that is directed against the host species of the primary antibody is applied. Dependent on the desired read-out method, the secondary detection antibody is specifically labeled with e.g. a fluorophore or an enzyme that can convert certain substrates to detectable products (such as colorimetric, chemiluminescence, or fluorescence). For example, the enzyme can be one of the following enzymes:
Horseradish Peroxide (HRP), often used for conjugation with a protein turns o-phenylenediamine dihydrochloride (OPD) into an amber product;
HRP turns 3,3′,5,5′-tetramethylbenzidine (TMB) into a blue product which becomes yellow in the presence of sulfuric or phosphoric acid;
HRP turns enhanced chemiluminescence (ECL) substrate into a product while emitting luminescence;
HRP turns 2,2′-Azinobis [3-ethylbenzothiazoline-6-sulfonic acid]-diammonium salt (ABTS) into a green product; and
Alkaline phosphatase turns p-nitrophenyl phosphate (PNPP) into a yellow product.

For the set-up of an indirect ELISA to determine the amount of antigen specific immunoglobulin in human serum after vaccination with a viral antigen many protocols are established. For example, a multi-well plate is coated with the viral target antigen and after sufficient incubation time washed and blocked. Then, serial diluted human serum and controls are added and sufficiently long incubated, allowing serum antibodies to bind to the antigen. Then, the plates are washed again and the labeled secondary antibody added and the plates sufficiently long incubated. After a final wash step, the substrate is added and the obtained colorimetric or luminescent result is read out. Finally, the obtained results are compared to the signal of a reference material leading to an arbitrary value, which needs to be converted to an absolute amount by quantitative mass spectrometry (qMS).

### Calibration of ELISA Result By Quantitative Mass Spectrometry (qMS)

The present application relates generally to calibration of ELISA result for immune response in human serum by quantitative mass spectrometry (qMS), which allows for the conversion the arbitrary read-out of ELISA to the absolute amount of antigen specific antibodies.

In one general aspect, the present application relates to a method of determining an absolute amount of antibodies specific to an antigen in a sample, the method comprises:
i. contacting a calibration material containing the antibodies with the antigen to form one or more protein complexes comprising the antigen and the antibodies bound to the antigen;
ii. determining the relative amount of the antibodies in the calibration material by an ELISA detection method;
iii. repeating step i by contacting the calibration material with the antigen to form one or more protein complexes comprising the antigen and the antibodies bound to the antigen;
iv. isolating the one or more protein complexes;
v. digesting the one or more protein complexes to generate a mixture comprising a proteotypic peptide of the antibodies;
vi. subjecting the mixture to quantitative mass spectrometry to measure a mass response of the proteotypic peptide;
vii. determining the absolute amount of the antibodies in the calibration material by comparing the mass response of the proteotypic peptide to a reference curve of the proteotypic peptide;
viii. establishing a conversion factor to correlate the relative amount of the antibodies in the calibration material as determined by the ELISA in step ii to the absolute amount of the antibodies in the calibration material as determined in step vii; and
ix. applying the conversion factor to determine the absolute amount of the antibodies in the sample.

In some embodiments, the method further comprises obtaining a reference curve of the proteotypic peptide before step i, wherein the reference curve relates a mass response of the proteotypic peptide to an absolute amount of the proteotypic peptide.

In some embodiments, in step vii, the absolute amount of the proteotypic peptide is first determined by comparing the mass response of the proteotypic peptide to the reference curve of the proteotypic peptide, and then the absolute amount of the antibodies in the calibration material is determined based on the absolute amount of the proteotypic peptide.

In some embodiments, the method further comprises:
x. contacting the sample with the antigen to form one or more protein complexes comprising the antigen and the antibodies bound to the antigen;
xi. determining the relative amount of the antibodies in the sample by the ELISA detection method; and
xii. determining the absolute amount of the antibodies in the sample by applying the conversion factor.

As used herein, the term “sample” refers to a sample taken from a subject who has been administered with a vaccine comprising the antigen, or from a subject who has not been vaccinated but has been infected with a virus comprising the antigen. For example, the subject can be a healthy participant or a patient who has been administered with a vaccine, or a healthy participant or a patient who has been in contact with the virus. Preferably, the patient has been or is infected with a virus such as respiratory syncytial virus (RSV), human immunodeficiency virus (HIV), influenza virus, Ebola virus, or hepatitis A, B, C or D virus. The vaccine can be a preventive vaccine or a therapeutic vaccine. Examples of the vaccine include, but are not limited to, a vaccine against a respiratory syncytial virus (RSV), a vaccine against a human immunodeficiency virus (HIV), a vaccine against an influenza virus, a vaccine against an Ebola virus, a vaccine against a hepatitis A, B, C or D virus, or a vaccine against SARS-COV-2 virus.

The sample can be a biological sample directly obtained from the subject. It can also be a processed sample of the biological sample, e.g., after the biological sample is centrifuged, frozen and thawed, etc.

In certain embodiments, the sample is a serum, plasma, or another biological fluid sample, preferably serum.

Preferably, the sample is an untested sample or unknown sample based on its ELISA response relative to the calibration material.

According to the embodiments of the present application, the calibration material is the reference material in the ELISA detection method. Examples of the ELISA can be direct ELISA, indirect ELISA, sandwich ELISA, reverse ELISA, or competition/inhibition ELISA.

In certain embodiments, the calibration material is a serum, plasma or another biological fluid sample, preferably serum.

According to the embodiments of the present application, the response of the reference material in the ELISA can be calibrated by quantitative mass spectrometry (qMS) to obtain an absolute amount of the antibodies. The qMS in the present application can be any mass spectrometry (MS)-based targeted protein quantification, which requires the use of one or more proteotypic peptides.

Proteotypic peptides are those peptides in a protein sequence that are most likely to be confidently observed by MS-based targeted proteomics methods. They are very useful for qualitative and quantitative identification of the protein of interest in a sample mixture interrogated by a tandem mass spectrometer. The selection of optimal proteotypic peptide for MS-based targeted protein quantification can be done by an empirically-driven approach or in silico analysis.

Traditionally, the proteotypic peptide in this application is a signature tryptic peptide, of which the amino acid sequence is (1) unique to polyclonal mixtures of antibodies, typically a part of the constant region of the protein, and (2) not found in any other protein that might be present in the plasma/serum (Halquist and Thomas Karnes, 2011).

LC-MS/MS-based assays for mAb quantification in plasma/serum have traditionally relied upon trypsin digestion of the mAb sample followed by quantification of a signature tryptic peptide. While the signature tryptic peptide approach ensures interference-free quantification of the mAb, the assays developed in this manner can only be applied to one analyte, and thus each new drug candidate requires the development of an entirely new assay to facilitate its quantification in pre-clinical animal studies.

According to the embodiments of the present application, the proteotypic peptide is a specific proteotypic peptide, which occurs only in the targeted antibodies, e.g., the antigen specific antibodies, when immune complexes are isolated. This type of proteotypic peptide is a universal surrogate peptide found in the Fc region of the majority of polyclonal mixtures of antibodies and Fc-fusion protein candidates. This approach, based upon a single universal surrogate peptide, is capable of quantifying a variety of polyclonal mixtures of antibodies and Fc-fusion protein candidates in plasma samples.

In certain embodiments, the specific proteotypic peptide occurs in the Fc region of human IgG, such as IgG1, IgG2, IgG3, or IgG4. Such peptide sequence would have the four general characteristics:
(1) The sequence would have to be present in human polyclonal mixtures of antibodies and Fc-fusion protein candidates, thus enabling its general applicability across development programs involving a variety of such protein candidates;
(2) The sequence would be reliably produced from trypsin digestion of human mAb and Fc-fusion protein analytes;
(3) The sequence would possess favorable LC-MS/MS characteristics such as good chromatographic peak shape, adequate chromatographic retention and efficient ionization; and
(4) The sequence would not be found in the protein sequences of any plasma/serum protein(s) present in any animal species typically used in pre-clinical drug development studies, thus ensuring quantification that is free of plasma protein-derived interferences (Furlong, Ouyang et al. 2012).

In certain embodiments, the preferred specific proteotypic peptides are listed in Table 1:

TABLE 1
Proteotypic peptide sequences

| SEQ ID | | |
|---|---|---|
| NO. | Sequence | Immunoglobulin |
| 1 | VVSVLTVLHQDWLNGK | IgG1, IgG3, IgG4 |
| 2 | VVSVLTVVHQDWLNGK | IgG2 |
| 3 | DTLMISR | all IgG |
| 4 | ALPAPIEK | IgG1, IgG3 |
| 5 | GLPAPIEK | IgG2 |
| 6 | GLPSSIEK | IgG4 |
| 7 | GFYPSDIAVEWESNGQ | IgG1, IgG4 |
| | PENNYK | |
| 8 | GFYPSDISVEWESNGQ | IgG2 |
| | PENNYK | |
| 9 | GFYPSDIAVEWESSGQ | IgG3 |
| | PENNYN | |
| 10 | VVSVLTVLHQDWLNGK | IgG1, IgG3, IgG4 |

In some embodiments, the antigen can be an RSV antigen, an HIV antigen, an influenza virus antigen, an Ebola virus antigen, or a hepatitis A, B, C or D virus antigen.

In certain embodiments, the antigen is an RSV-B antigen, preferably an RSV-B F antigen.

In certain embodiments, the antigen is an HIV antigen, preferably a Mosaic gp140.

In certain embodiments, the antigen is a SARS-COV-2 antigen, preferably a spike antigen (SARS-COV-2 S).

In some embodiments, the antibodies in the sample bind to the same or different epitopes of the antigen.

In some embodiments, the antibodies comprise one or more of immunoglobulin G (IgG), such as IgG1, IgG2, IgG3, or IgG4. Preferably, these antibodies comprise one of the proteotypic peptides listed in Table 1.

In some embodiments, the step iv further comprises one or more steps of denaturing, reducing, alkylating and/or diluting the one or more protein complexes.

In some embodiments, the method further comprises subjecting the mixture to liquid chromatography (LC) prior to step vi, preferably the LC is high performance liquid chromatogram (HPLC).

In some embodiments, the quantitative mass spectrometry is a tandem mass spectrometry. Preferably, the quantitative mass spectrometry comprises multiple reaction monitoring (MRM) or selected reaction monitoring (SRM) or a parallel reaction monitoring (PRM).

In some embodiments, the method comprises measuring the mass response of the intact mass of the proteotypic peptide.

In some embodiments, the method comprises measuring the mass response of the fragment mass of the proteotypic peptide.

In some embodiments, the method further comprises comparing the mass response of the intact mass of the proteotypic peptide to the reference curve of the proteotypic peptide.

In some embodiments, the method further comprises comparing the mass response of the fragment mass of the proteotypic peptide to the reference curve of the proteotypic peptide.

According to the embodiments of the present application, the absolute amount of the antibodies specific to the antigen is determined by comparing the mass response of the proteotypic peptide to a reference curve of the proteotypic peptide.

The reference curve of the proteotypic peptide can be obtained by any known method in the art. For example, to establish such a reference curve, the calibration material containing different amounts of the proteotypic peptide are obtained, either by dilution of the same calibration material or by spiking different amount of proteotypic peptide in the calibration material. For each amount/concentration of the calibration material, the mass response of the proteotypic peptide is determined according to the methods of the present application. Then, the absolute amounts of the proteotypic peptide are plotted against mass response to obtain a reference curve of the proteotypic peptide. Hence, the reference curve relates the mass response of the proteotypic peptide to the absolute amount of the proteotypic peptide specific to the antigen.

According to the embodiments of the present application, when determining the absolute amount of the antibodies specific to the antigen, the absolute amount of the proteotypic peptide is first determined, by comparing the mass response of the proteotypic peptide to a reference curve of the proteotypic peptide. Then, the absolute amount of the antibodies specific to the antigen is determined based on the 1) absolute amount of the proteotypic peptide, 2) the mole ratio or weight ratio between the antibody and the corresponding proteotypic peptide, and 3) the dilution factor of the serum used in the ELISA.

In some embodiments, the absolute amount of the antibodies in the calibration material can be expressed in molar concentration or weight concentration.

According to the embodiments of the present application, the relative amount of the antibodies in the calibration material is determined by an ELISA in step ii. Examples of the ELISA include, but are not limited to, direct ELISA, indirect ELISA, sandwich ELISA, reverse ELISA, or competition/inhibition ELISA.

In some embodiments, a conversion factor is established to correlate the relative amount of the antibodies as determined by the ELISA in step ii to the absolute amount of the antibodies as determined in step vii. In some embodiments, the conversion factors can be expressed as a formula or a plot or a curve, which relates the relative amount of the antibodies to the absolute amount of the antibodies.

After the establishment of the conversion factor, the relative amount of the antibodies is converted to an absolute amount by applying the conversion factor to the relative amount. The absolute amount of the antibodies in the sample can be expressed in molar concentration or weight concentration, dependent of the expression of the absolute amount of the antibodies in the reference material. For example, if the absolute amount of the antibodies in the reference material determined is expressed in molar concentration, the absolute amount of the antibodies in the sample is also expressed in molar concentration; if the absolute amount of the antibodies in the reference material is expressed in weight concentration, the absolute amount of the antibodies in the sample is also expressed in weight concentration.

In some embodiments, the absolute amount of the antibodies in the sample is determined by directly comparing the mass response of the proteotypic peptide to a reference curve of the proteotypic peptide, which indirectly relates the absolute amount of the antibodies to the mass response of the proteotypic peptide determined by qMS.

In some embodiments, the reference curve is a sigmoidal curve. In certain embodiments, the absolute amount of antibodies is determined by direct comparison to the reference curve or by sigmoidal curve fitting.

In some embodiments, wherein the absolute amount of the proteotypic peptide is determined by comparing the mass response of the proteotypic peptide to the reference curve of the proteotypic peptide, and then the absolute amount of the antibodies is determined based on the 1) absolute amount of the proteotypic peptide, 2) the mole ratio or weight ratio between the antibody and the corresponding proteotypic peptide, and 3) the dilution factor of the serum used in the ELISA.

In some embodiments, more than one proteotypic peptides can be used in the method. In further embodiments, the absolute amount of the antibodies specific to the antigen in the sample is the sum of the absolute amount of the antibodies IgG1 ,2, 3, and 4.

For example, a method can use a proteotypic peptide having the amino acid sequence of SEQ ID NO. 1 and a proteotypic peptide having the amino acid sequence of SEQ ID NO. 2. Then, the absolute amount of the antibodies IgG1, 3, and 4 is determined based on the reference curve of the proteotypic peptide having the amino acid sequence of SEQ ID NO. 1, and the absolute amount of the antibodies IgG2 is determined based on the reference curve of the proteotypic peptide having the amino acid sequence of SEQ ID NO. 2. Then, the absolute amount of the antibodies specific to the antigen is the sum of the absolute amount of the antibodies IgG1, 3, and 4 and the absolute amount of the antibodies IgG2.

In another general aspect, the present application also relates to a method of determining an absolute amount of antibodies specific to an RSV antigen in a sample from a subject administered with a vaccine comprising the RSV antigen, the method comprising:
i. contacting the sample with the RSV antigen to form one or more protein complexes comprising the RSV antigen and the antibodies bound to the antigen;
ii. isolating the one or more protein complexes;
iii. digesting the one or more protein complexes to generate a first mixture comprising a proteotypic peptide having the amino acid sequence of SEQ ID NO. 1;
iv. subjecting the first mixture to liquid chromatography (LC), preferably high performance liquid chromatogram (HPLC), to generate a second mixture;
v. subjecting the second mixture to a quantitative mass spectrometry to thereby generate a mass spectrum of the proteotypic peptide;
vi. measuring a mass response of the proteotypic peptide from the mass spectrum; and
vii. determining the absolute amount of the antibodies specific to the RSV antigen in the sample by comparing the mass response of the proteotypic peptide to a reference curve of the proteotypic peptide.

In some embodiments, the method further comprises obtaining a reference curve of the proteotypic peptide before step i, wherein the reference curve relates a mass response of the proteotypic peptide to an absolute amount of the proteotypic peptide.

In some embodiments, wherein in step vii, the absolute amount of the proteotypic peptide is first determined by comparing the mass response of the proteotypic peptide to the reference curve of the proteotypic peptide, and then the absolute amount of the antibodies specific to the RSV antigen is determined based on the absolute amount of the proteotypic peptide.

In some embodiments, the sample is a serum, plasma or another biological fluid sample, preferably serum.

In certain embodiments, the sample is a processed sample of the biological sample, e.g., after the biological sample is centrifuged, frozen and thawed, etc.

In some embodiments, the RSV vaccine is a preventive vaccine or a therapeutic vaccine.

According to the embodiments of the present application, the calibration material is the reference material in the ELISA detection method. Examples of the ELISA can be direct ELISA, indirect ELISA, sandwich ELISA, reverse ELISA, or competition/inhibition ELISA.

In some embodiments, the antibodies in the sample bind to the same or different epitopes of the RSV antigen.

In certain embodiments, the RSV antigen is an RSV-B antigen, preferably an RSV-B F antigen.

In some embodiments, the antibodies comprise one or more of immunoglobulin G (IgG), such as IgG1, IgG2, IgG3, or IgG4.

In some embodiments, the proteotypic peptide occurs to the Fc region of the antibodies.

In some embodiments, the proteotypic peptide comprises the amino acid sequence of SEQ ID NO. 1.

In some embodiments, the step ii further comprises one or more steps of denaturing, reducing, alkylating and/or diluting the one or more protein complexes.

In some embodiments, the quantitative mass spectrometry (qMS) is a tandem mass spectrometry. Preferably, the quantitative mass spectrometry comprises multiple reaction monitoring (MRM) or selected reaction monitoring (SRM) or a parallel reaction monitoring (PRM).

In some embodiments, the step vi of the method further comprises measuring the mass response of the intact mass of the proteotypic peptide.

In some embodiments, the step vi of the method further comprises measuring the mass response of the fragment mass of the proteotypic peptide.

In some embodiments, the method further comprises comparing the mass response of the intact mass of the proteotypic peptide to a reference curve of the proteotypic peptide.

In some embodiments, the method further comprises comparing the mass response of the fragment mass of the proteotypic peptide to a reference curve of the proteotypic peptide.

In another general aspect, the present application also relates to a method of determining an absolute amount of antibodies specific to an HIV antigen in a sample from a subject administered with a vaccine comprising the HIV antigen, the method comprising:
i. contacting the sample with the HIV antigen to form one or more protein complexes comprising the HIV antigen and the antibodies bound to the antigen;
ii. isolating the one or more protein complexes;
iii. digesting the one or more protein complexes to generate a first mixture comprising a proteotypic peptide having the amino acid sequence of SEQ ID NO. 1 and a proteotypic peptide having the amino acid sequence of SEQ ID NO. 2;
iv. subjecting the first mixture to liquid chromatography (LC), preferably high performance liquid chromatogram (HPLC), to generate a second mixture;
v. subjecting the second mixture to a quantitative mass spectrometry to thereby generate a mass spectrum of each of the proteotypic peptides;
vi. measuring a mass response of each of the proteotypic peptides from the mass spectrum; and
vii. determining the absolute amount of the antibodies specific to the HIV antigen in the sample by comparing the mass response of each of the proteotypic peptides to a reference curve of each of the proteotypic peptides, wherein the reference curve relates a mass spectrometric response of the proteotypic peptide to an absolute amount of the proteotypic peptide.

In some embodiments, the method further comprises obtaining a reference curve of each of the proteotypic peptides before step i.

In some embodiments, wherein in step vii, the absolute amount of the proteotypic peptide having the amino acid sequence of SEQ ID NO. 1 is determined by comparing the mass response of the said proteotypic peptide to the reference curve of the said proteotypic peptide, and then the absolute amount of the antibodies IgG1, 3, and 4 specific to the HIV antigen is determined based on the absolute amount of the said proteotypic peptide.

In some embodiments, wherein in step vii, the absolute amount of the proteotypic peptide having the amino acid sequence of SEQ ID NO. 2 is determined by comparing the mass response of the said proteotypic peptide to the reference curve of the said proteotypic peptide, and then the absolute amount of the antibodies IgG2 specific to the HIV antigen is determined based on the absolute amount of the said proteotypic peptide.

In further embodiments, the absolute amount of the antibodies in the sample is the sum of the absolute amount of the antibodies IgG1, 3, and 4 and the absolute amount of the antibodies IgG2.

In some embodiments, the sample is a serum, plasma or another biological fluid sample, preferably serum.

In certain embodiments, the sample is a processed sample of the biological sample, e.g., after the biological sample is centrifuged, frozen and thawed, etc.

In some embodiments, the HIV vaccine is a preventive vaccine or a therapeutic vaccine.

According to the embodiments of the present application, the calibration material is the reference material in the ELISA detection method. Examples of the ELISA can be direct ELISA, indirect ELISA, sandwich ELISA, reverse ELISA, or competition/inhibition ELISA.

In some embodiments, the antibodies in the sample bind to the same or different epitopes of the HIV antigen.

In certain embodiments, the HIV antigen is Mosaic gp140.

In some embodiments, the antibodies comprise one or more of immunoglobulin G (IgG), such as IgG1, IgG2, IgG3, or IgG4.

In some embodiments, the proteotypic peptide occurs to the Fc region of the antibodies.

In some embodiments, the step ii further comprises one or more steps of denaturing, reducing, alkylating and/or diluting the one or more protein complexes.

In some embodiments, the quantitative mass spectrometry (qMS) is a tandem mass spectrometry. Preferably, the quantitative mass spectrometry comprises multiple reaction monitoring (MRM) or selected reaction monitoring (SRM) or a parallel reaction monitoring (PRM).

In some embodiments, the step vi of the method further comprises measuring the mass response of the intact mass of each of the proteotypic peptides.

In some embodiments, the step vi of the method further comprises measuring the mass response of the fragment mass of each of the proteotypic peptides.

In some embodiments, the method further comprises comparing the mass response of each of the intact mass of the proteotypic peptides to a reference curve of the corresponding proteotypic peptide.

In some embodiments, the method further comprises comparing the mass response of the fragment mass of each of the proteotypic peptides to a reference curve of the corresponding proteotypic peptide.

In another general aspect, the present application also relates to a method of determining an absolute amount of antibodies specific to a SARS-COV-2 antigen in a sample from a subject administered with a vaccine comprising the SARS-COV-2 antigen, the method comprising:
i. contacting the sample with the SARS-COV-2 antigen to form one or more protein complexes comprising the SARS-COV-2 antigen and the antibodies bound to the antigen;
ii. isolating the one or more protein complexes;
iii. digesting the one or more protein complexes to generate a first mixture comprising a proteotypic peptide having the amino acid sequence of SEQ ID NO. 1 and a proteotypic peptide having the amino acid sequence of SEQ ID NO. 2;
iv. subjecting the first mixture to liquid chromatography (LC), preferably high performance liquid chromatogram (HPLC), to generate a second mixture;
v. subjecting the second mixture to a quantitative mass spectrometry to thereby generate a mass spectrum of each of the proteotypic peptides;
vi. measuring a mass response of each of the proteotypic peptides from the mass spectrum; and
vii. determining the absolute amount of the antibodies specific to the SARS-COV-2 antigen in the sample by comparing the mass response of each of the proteotypic peptides to a reference curve of each of the proteotypic peptides, wherein the reference curve relates a mass spectrometric response of the proteotypic peptide to an absolute amount of the proteotypic peptide.

In some embodiments, the method further comprises obtaining a reference curve of each of the proteotypic peptides before step i.

In some embodiments, wherein in step vii, the absolute amount of the proteotypic peptide having the amino acid sequence of SEQ ID NO. 1 is determined by comparing the mass response of the said proteotypic peptide to the reference curve of the said proteotypic peptide, and then the absolute amount of the antibodies IgG1, 3, and 4 specific to the SARS-COV-2 antigen is determined based on the absolute amount of the said proteotypic peptide.

In some embodiments, wherein in step vii, the absolute amount of the proteotypic peptide having the amino acid sequence of SEQ ID NO. 2 is determined by comparing the mass response of the said proteotypic peptide to the reference curve of the said proteotypic peptide, and then the absolute amount of the antibodies IgG2 specific to the SARS-COV-2 antigen is determined based on the absolute amount of the said proteotypic peptide.

In further embodiments, the absolute amount of the antibodies in the sample is the sum of the absolute amount of the antibodies IgG1, 3, and 4 and the absolute amount of the antibodies IgG2.

In some embodiments, the sample is a serum, plasma or another biological fluid sample, preferably serum.

In certain embodiments, the sample is a processed sample of the biological sample, e.g., after the biological sample is centrifuged, frozen and thawed, etc.

In some embodiments, the SARS-COV-2 vaccine is a preventive vaccine or a therapeutic vaccine.

According to the embodiments of the present application, the calibration material is the reference material in the ELISA detection method. Examples of the ELISA can be direct ELISA, indirect ELISA, sandwich ELISA, reverse ELISA, or competition/inhibition ELISA.

In some embodiments, the antibodies in the sample bind to the same or different epitopes of the SARS-COV-2 antigen.

In certain embodiments, the SARS-COV-2 antigen is SARS-COV-2 S.

In some embodiments, the antibodies comprise one or more of immunoglobulin G (IgG), such as IgG1, IgG2, IgG3, or IgG4.

In some embodiments, the proteotypic peptide occurs to the Fc region of the antibodies.

In some embodiments, the step ii further comprises one or more steps of denaturing, reducing, alkylating and/or diluting the one or more protein complexes.

In some embodiments, the quantitative mass spectrometry (qMS) is a tandem mass spectrometry. Preferably, the quantitative mass spectrometry comprises multiple reaction monitoring (MRM) or selected reaction monitoring (SRM) or a parallel reaction monitoring (PRM).

In some embodiments, the step vi of the method further comprises measuring the mass response of the intact mass of each of the proteotypic peptides.

In some embodiments, the step vi of the method further comprises measuring the mass response of the fragment mass of each of the proteotypic peptides.

In some embodiments, the method further comprises comparing the mass response of each of the intact mass of the proteotypic peptides to a reference curve of the corresponding proteotypic peptide.

In some embodiments, the method further comprises comparing the mass response of the fragment mass of each of the proteotypic peptides to a reference curve of the corresponding proteotypic peptide.

### EMBODIMENTS

The invention provides also the following non-limiting embodiments.

Embodiment 1 is a method of determining an absolute amount of antibodies specific to an antigen in a sample, the method comprising:
i. contacting a calibration material containing the antibodies with the antigen to form one or more protein complexes comprising the antigen and the antibodies bound to the antigen;
ii. determining the relative amount of the antibodies in the calibration material by an ELISA detection method;
iii. repeating step i by contacting the calibration material with the antigen to form one or more protein complexes comprising the antigen and the antibodies bound to the antigen;
iv. isolating the one or more protein complexes;
v. digesting the one or more protein complexes to generate a mixture comprising a proteotypic peptide of the antibodies;
vi. subjecting the mixture to quantitative mass spectrometry to measure a mass response of the proteotypic peptide;
vii. determining the absolute amount of the antibodies in the calibration material by comparing the mass response of the proteotypic peptide to a reference curve of the proteotypic peptide;
viii. establishing a conversion factor to correlate the relative amount of the antibodies in the calibration material as determined by the ELISA in step ii to the absolute amount of the antibodies in the calibration material as determined in step vii; and
ix. applying the conversion factor to determine the absolute amount of the antibodies in the sample.

Embodiment 1a is the method of embodiment 1, further comprising obtaining a reference curve of the proteotypic peptide before step i, wherein the reference curve relates a mass response of the proteotypic peptide to an absolute amount of the proteotypic peptide.

Embodiment 1b is the method of embodiment 1 or 1a, wherein in step vii, an absolute amount of the proteotypic peptide is first determined by comparing the mass response of the proteotypic peptide to the reference curve of the proteotypic peptide, and then the absolute amount of the antibodies in the calibration material is determined based on the absolute amount of the proteotypic peptide.

Embodiment 1c is the method of embodiment 1 or 1a or 1b, further comprising:
x. contacting the sample with the antigen to form one or more protein complexes comprising the antigen and the antibodies bound to the antigen;
xi. determining the relative amount of the antibodies in the sample by the ELISA detection method; and
xii. determining the absolute amount of the antibodies in the sample by applying the conversion factor.

Embodiment 2 is the method of any one of embodiments 1-1c, wherein the sample is obtained from a subject administered with a vaccine comprising the antigen, or from a subject who has been in contact with a virus comprising the antigen.

Embodiment 2a is the method of embodiment 2, wherein the subject is a healthy participant or a patient.

Embodiment 2b is the method of embodiment 2a, wherein the patient is infected with a virus selected from the group consisting of respiratory syncytial virus (RSV), human immunodeficiency virus (HIV), influenza virus, Ebola virus, hepatitis A, B, C or D virus, and a SARS-COV-2 virus.

Embodiment 3 is the method of any one of embodiments 2-2b, wherein the vaccine is a preventive vaccine or a therapeutic vaccine.

Embodiment 3a is the method of embodiment 3, wherein the vaccine is against a respiratory syncytial virus (RSV), a human immunodeficiency virus (HIV), an influenza virus, an Ebola virus, against a hepatitis A, B, C or D virus, or against a SARS-COV-2 virus.

Embodiment 4 is the method of any one of embodiments 1-3a, wherein the antigen is selected from the group consisting of RSV antigen, HIV antigen, influenza virus antigen, Ebola virus antigen, hepatitis A antigen, hepatitis B antigen, hepatitis C antigen, hepatitis D antigen, and SARS-COV-2 antigen.

Embodiment 4a is the method of embodiment 4, wherein the antigen is an RSV-B antigen, preferably an RSV-B F antigen.

Embodiment 4b is the method of embodiment 4, wherein the antigen is an HIV antigen, preferably gp140 or gp120, more preferably gp140 Clade C.

Embodiment 4c is the method of embodiment 4, wherein the antigen is a SARS-COV-2 antigen, preferably a spike antigen (SARS-COV-2 S).

Embodiment 5 is the method of any one of embodiments 1-4c, wherein the antibodies comprise one or more of immunoglobulin G (IgG).

Embodiment 5a is the method of embodiment 5, wherein the IgG is IgG1, IgG2, IgG3 or IgG4.

Embodiment 5b is the method of embodiment 5, wherein the antibodies in the sample bind to the same or different epitopes of the antigen.

Embodiment 6 is the method of any one of embodiments 1-5b, wherein the proteotypic peptide occurs in the antibodies specific to the antigen.

Embodiment 6a is the method of any one of embodiments 1-6, wherein the proteotypic peptide occurs in the Fc region of the antibodies specific to the antigen.

Embodiment 6b is the method of any one of embodiments 1-6a, wherein the proteotypic peptide comprises the amino acid sequence selected from the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, and SEQ ID NO. 10.

Embodiment 7 is the method of any one of embodiments 1-6b, wherein the sample is a serum, plasma or another biological fluid sample, preferably serum.

Embodiment 7a is the method of embodiment 7, wherein the sample is a processed sample of the biological sample.

Embodiment 7b is the method of embodiment 7a, wherein the biological sample is centrifuged, frozen and/or thawed.

Embodiment 8 is the method of any one of embodiments 1-7b, wherein the calibration material is a serum, plasma or another biological fluid sample.

Embodiment 9 is the method of any one of embodiments 1-8, wherein the step iv further comprises one or more steps of denaturing, reducing, alkylating and/or diluting the one or more protein complexes.

Embodiment 9a is the method of any one of embodiments 1-9, further comprising subjecting the mixture to liquid chromatography (LC) prior to step iv, preferably the LC is high performance liquid chromatogram (HPLC).

Embodiment 10 is the method of any one of embodiments 1-9a, wherein the quantitative mass spectrometry is a tandem mass spectrometry.

Embodiment 10a is the method of any one of embodiments 1-10, wherein the quantitative mass spectrometry comprises multiple reaction monitoring (MRM).

Embodiment 10b is the method of any one of embodiments 1-10, wherein the quantitative mass spectrometry comprises selected reaction monitoring (SRM).

Embodiment 10c is the method of any one of embodiments 1-10, wherein the quantitative mass spectrometry comprises a parallel reaction monitoring (PRM).

Embodiment 10d is the method of embodiment 10c, wherein the PRM comprises a C18 nano-LC connected to an ESI-Q-Orbitrap mass spectrometer.

Embodiment 11 is the method of any one of embodiments 1-10c, further comprising measuring the mass response of the intact mass of the proteotypic peptide.

Embodiment 11a is the method of embodiment 11, further comprising comparing the mass response of the intact mass of the proteotypic peptide to the reference curve of the proteotypic peptide.

Embodiment 11c is the method of any one of embodiments 1-10c, further comprising measuring the mass response of the fragment mass of the proteotypic peptide.

Embodiment 11d is the method of embodiment 11c, further comprising comparing the mass response of the fragment mass of the proteotypic peptide to the reference curve of the proteotypic peptide.

Embodiment 12 is the method of any one of embodiments 1-11d, wherein the absolute amount of the antibodies in the calibration material is expressed in weight concentration or molar concentration.

Embodiment 12a is the method of any one of embodiments 1-12, wherein the absolute amount of the antibodies in the sample is expressed in weight concentration or molar concentration.

Embodiment 13 is a method of determining an absolute amount of antibodies specific to an RSV antigen in a sample from a subject administered with a vaccine comprising the RSV antigen, the method comprising:
i. contacting the sample with the RSV antigen to form one or more protein complexes comprising the RSV antigen and the antibodies bound to the antigen;
ii. isolating the one or more protein complexes;
iii. digesting the one or more protein complexes to generate a first mixture comprising a proteotypic peptide having the amino acid sequence of SEQ ID NO. 1;
iv. subjecting the first mixture to liquid chromatography (LC), preferably high performance liquid chromatogram (HPLC), to generate a second mixture;
v. subjecting the second mixture to a quantitative mass spectrometry to thereby generate a mass spectrum of the proteotypic peptide;
vi. measuring a mass response of the proteotypic peptide from the mass spectrum; and
vii. determining the absolute amount of the antibodies specific to the RSV antigen in the sample by comparing the mass response of the proteotypic peptide to a reference curve of the proteotypic peptide.

Embodiment 14 is the method of embodiment 13, further comprising obtaining a reference curve of the proteotypic peptide before step i, wherein the reference curve relates a mass response of the proteotypic peptide to an absolute amount of the proteotypic peptide.

Embodiment 14a is the method of embodiment 14 or 14a, wherein the subject is a healthy participant or a patient.

Embodiment 14b is the method of embodiment 14a, wherein the patient is an RSV infected patient.

Embodiment 14c is the method of any one of embodiments 13-14b, wherein in step vii, an absolute amount of the proteotypic peptide is first determined by comparing the mass response of the proteotypic peptide to the reference curve of the proteotypic peptide, and then the absolute amount of the antibodies specific to the RSV antigen is determined based on the absolute amount of the proteotypic peptide.

Embodiment 15 is the method of any one of embodiments 13-14c, wherein the vaccine is a preventive vaccine or a therapeutic vaccine.

Embodiment 16 is the method of any one embodiments 13-15, wherein the antigen is an RSV-B antigen, preferably RSV-B F antigen.

Embodiment 17 is the method of any one of embodiments 13-16, wherein the antibodies comprise one or more of immunoglobulin G (IgG).

Embodiment 17a is the method of embodiment 17, wherein the IgG is IgG1, IgG2, IgG3 or IgG4.

Embodiment 17b is the method of embodiment 17, wherein the antibodies in the sample bind to the same or different epitopes of the RSV antigen.

Embodiment 18 is the method of any one of embodiments 13-17b, wherein the proteotypic peptide occurs in the antibodies specific to the RSV antigen.

Embodiment 18a is the method of any one of embodiments 13-18, wherein the proteotypic peptide occurs in the Fc region of antibodies specific to the RSV antigen.

Embodiment 19 is the method of any one of embodiments 13-18a, wherein the sample is a serum, plasma or another biological fluid sample, preferably serum. Embodiment 19a is the method of embodiment 19, wherein the sample is a processed sample of the biological sample.

Embodiment 19b is the method of embodiment 19a, wherein the biological sample is centrifuged, frozen and/or thawed.

Embodiment 20 is the method of any one of embodiments 13-19b, wherein the step ii further comprises one or more steps of denaturing, reducing, alkylating and/or diluting the one or more protein complexes.

Embodiment 21 is the method of any one of embodiments 13-20, wherein the quantitative mass spectrometry is a tandem mass spectrometry.

Embodiment 21a is the method of any one of embodiments 13-21, wherein the quantitative mass spectrometry comprises multiple reaction monitoring (MRM).

Embodiment 21b is the method of any one of embodiments 13-21, wherein the quantitative mass spectrometry comprises selected reaction monitoring (SRM).

Embodiment 21c is the method of any one of embodiments 13-21, wherein the quantitative mass spectrometry comprises a parallel reaction monitoring (PRM).

Embodiment 21d is the method of embodiment 21c, wherein the PRM comprises a C18 nano-LC connected to an ESI-Q-Orbitrap mass spectrometer.

Embodiment 22 is the method of any one of embodiments 13-21d, further comprising measuring the mass response of the intact mass of the proteotypic peptide.

Embodiment 22a is the method of embodiment 22, further comprising comparing the mass response of the intact mass of the proteotypic peptide to the reference curve of the proteotypic peptide.

Embodiment 22b is the method of any one of embodiments 13-21d, further comprising measuring the mass response of the fragment mass of the proteotypic peptide.

Embodiment 22c is the method of embodiment 22b, further comprising comparing the mass response of the fragment mass of the proteotypic peptide to the reference curve of the proteotypic peptide.

Embodiment 23 is the method of any one of embodiments 13-22c, wherein the absolute amount of the antibodies in the sample is expressed in weight concentration or molar concentration.

Embodiment 24 is a method of determining an absolute amount of antibodies specific to an HIV antigen in a sample from a subject administered with a vaccine comprising the HIV antigen, the method comprising:
i. contacting the sample with the HIV antigen to form one or more protein complexes comprising the HIV antigen and the antibodies bound to the antigen;
ii. isolating the one or more protein complexes;
iii. digesting the one or more protein complexes to generate a first mixture comprising a proteotypic peptide having the amino acid sequence of SEQ ID NO. 1 and a proteotypic peptide having the amino acid sequence of SEQ ID NO. 2;
iv. subjecting the first mixture to liquid chromatography (LC), preferably high performance liquid chromatogram (HPLC), to generate a second mixture;
v. subjecting the second mixture to a quantitative mass spectrometry to thereby generate a mass spectrum of each of the proteotypic peptides;
vi. measuring a mass response of each of the proteotypic peptides from the mass spectrum; and
vii. determining the absolute amount of the antibodies specific to the HIV antigen in the sample by comparing the mass response of each of the proteotypic peptides to a reference curve of each of the proteotypic peptides, wherein the reference curve relates a mass spectrometric response of the proteotypic peptide to an absolute amount of the proteotypic peptide.

Embodiment 24 is the method of embodiment 23, further comprising obtaining a reference curve of each of the proteotypic peptides before step i.

Embodiment 25 is the method of embodiment 23 or 24, wherein in step vii, an absolute amount of the proteotypic peptide having the amino acid sequence of SEQ ID NO. 1 is determined by comparing the mass response of the said proteotypic peptide to the reference curve of the said proteotypic peptide, and then an absolute amount of antibodies IgG1, 3, and 4 specific to the HIV antigen is determined based on the absolute amount of the said proteotypic peptide.

Embodiment 25a is the method of any one of embodiments 23-25, wherein in step vii, an absolute amount of the proteotypic peptide having the amino acid sequence of SEQ ID NO. 2 is determined by comparing the mass response of the said proteotypic peptide to the reference curve of the said proteotypic peptide, and then an absolute amount of antibodies IgG2 specific to the HIV antigen is determined based on the absolute amount of the said proteotypic peptide.

Embodiment 25b is the method of any one of embodiments 23-25a, wherein the absolute amount of the antibodies in the sample is the sum of the absolute amount of the antibodies IgG1, 3, and 4 and the absolute amount of the antibodies IgG2.

Embodiment 26 is the method of any one of embodiments 23-25b, wherein the subject is a healthy participant or a patient.

Embodiment 26a is the method of embodiment 26, wherein the patient is an HIV infected patient.

Embodiment 27 is the method of any one of embodiments 23-26a, wherein the vaccine is a preventive vaccine or a therapeutic vaccine.

Embodiment 28 is the method of any one embodiments 23-27, wherein the antigen is Mosaic gp140.

Embodiment 29 is the method of any one of embodiments 23-28, wherein the antibodies comprise one or more of immunoglobulin G (IgG).

Embodiment 29a is the method of embodiment 29, wherein the IgG is IgG1, IgG2, IgG3 or IgG4.

Embodiment 29b is the method of embodiment 29, wherein the antibodies in the sample bind to the same or different epitopes of the HIV antigen.

Embodiment 30 is the method of any one of embodiments 23-29b, wherein the proteotypic peptide occurs in the antibodies specific to the HIV antigen.

Embodiment 30a is the method of any one of embodiments 23-30, wherein the proteotypic peptide occurs in the Fc region of antibodies specific to the HIV antigen.

Embodiment 31 is the method of any one of embodiments 23-30a, wherein the sample is a serum, plasma or another biological fluid sample, preferably serum.

Embodiment 31a is the method of embodiment 30, wherein the sample is a processed sample of the biological sample.

Embodiment 31b is the method of embodiment 31a, wherein the biological sample is centrifuged, frozen and/or thawed.

Embodiment 32 is the method of any one of embodiments 23-31b, wherein the step ii further comprises one or more steps of denaturing, reducing, alkylating and/or diluting the one or more protein complexes.

Embodiment 33 is the method of any one of embodiments 23-32, wherein the quantitative mass spectrometry is a tandem mass spectrometry.

Embodiment 33a is the method of any one of embodiments 23-33, wherein the quantitative mass spectrometry comprises multiple reaction monitoring (MRM).

Embodiment 33b is the method of any one of embodiments 23-33, wherein the quantitative mass spectrometry comprises selected reaction monitoring (SRM).

Embodiment 33c is the method of any one of embodiments 23-33, wherein the quantitative mass spectrometry comprises a parallel reaction monitoring (PRM).

Embodiment 33d is the method of embodiment 33c, wherein the PRM comprises a C18 nano-LC connected to an ESI-Q-Orbitrap mass spectrometer.

Embodiment 34 is the method of any one of embodiments 23-33d, further comprising measuring the mass response of the intact mass of each of the proteotypic peptides.

Embodiment 34a is the method of embodiment 34, further comprising comparing the mass response of the intact mass of each of the proteotypic peptides to the reference curve of the corresponding proteotypic peptide.

Embodiment 34b is the method of any one of embodiments 23-33d, further comprising measuring the mass response of the fragment mass of each of the proteotypic peptides.

Embodiment 34c is the method of embodiment 34b, further comprising comparing the mass response of the fragment mass of each of the proteotypic peptides to the reference curve of the corresponding proteotypic peptide.

Embodiment 35 is the method of any one of embodiments 23-34c, wherein the absolute amount of the antibodies in the sample is expressed in weight concentration or molar concentration.

Embodiment 36 is a method of determining an absolute amount of antibodies specific to a SARS-COV-2 antigen in a sample from a subject administered with a vaccine comprising the SARS-COV-2 antigen, the method comprising:
i. contacting the sample with the SARS-COV-2 antigen to form one or more protein complexes comprising the SARS-COV-2 antigen and the antibodies bound to the antigen;
ii. isolating the one or more protein complexes;
iii. digesting the one or more protein complexes to generate a first mixture comprising a proteotypic peptide having the amino acid sequence of SEQ ID NO. 1 and a proteotypic peptide having the amino acid sequence of SEQ ID NO. 2;
iv. subjecting the first mixture to liquid chromatography (LC), preferably high performance liquid chromatogram (HPLC), to generate a second mixture;
v. subjecting the second mixture to a quantitative mass spectrometry to thereby generate a mass spectrum of each of the proteotypic peptides;
vi. measuring a mass response of each of the proteotypic peptides from the mass spectrum; and
vii. determining the absolute amount of the antibodies specific to the SARS-COV-2 antigen in the sample by comparing the mass response of each of the proteotypic peptides to a reference curve of each of the proteotypic peptides, wherein the reference curve relates a mass spectrometric response of the proteotypic peptide to an absolute amount of the proteotypic peptide.

Embodiment 37 is the method of embodiment 36, further comprising obtaining a reference curve of each of the proteotypic peptides before step i.

Embodiment 38 is the method of embodiment 36 or 37, wherein in step vii, an absolute amount of the proteotypic peptide having the amino acid sequence of SEQ ID NO. 1 is determined by comparing the mass response of the said proteotypic peptide to the reference curve of the said proteotypic peptide, and then an absolute amount of antibodies IgG1, 3, and 4 specific to the SARS-COV-2 antigen is determined based on the absolute amount of the said proteotypic peptide.

Embodiment 38a is the method of any one of embodiments 36-38, wherein in step vii, an absolute amount of the proteotypic peptide having the amino acid sequence of SEQ ID NO. 2 is determined by comparing the mass response of the said proteotypic peptide to the reference curve of the said proteotypic peptide, and then an absolute amount of antibodies IgG2 specific to the SARS-COV-2 antigen is determined based on the absolute amount of the said proteotypic peptide.

Embodiment 38b is the method of any one of embodiments 36-38a, wherein the absolute amount of the antibodies in the sample is the sum of the absolute amount of the antibodies IgG1, 3, and 4 and the absolute amount of the antibodies IgG2.

Embodiment 39 is the method of any one of embodiments 36-38b, wherein the subject is a healthy participant or a patient.

Embodiment 39a is the method of embodiment 39, wherein the patient is a SARS-COV-2 infected patient.

Embodiment 39b is the method of any one of embodiments 36-39a, wherein the antigen is SARS-COV-2 S.

Embodiment 40 is the method of any one of embodiments 36-39b, wherein the vaccine is a preventive vaccine or a therapeutic vaccine.

Embodiment 41 is the method of any one of embodiments 36-40, wherein the antibodies comprise one or more of immunoglobulin G (IgG).

Embodiment 41a is the method of embodiment 41, wherein the IgG is IgG1, IgG2, IgG3 or IgG4.

Embodiment 41b is the method of embodiment 41, wherein the antibodies in the sample bind to the same or different epitopes of the SARS-COV-2 antigen.

Embodiment 42 is the method of any one of embodiments 36-41b, wherein the proteotypic peptide occurs in the antibodies specific to the SARS-COV-2 antigen.

Embodiment 42a is the method of any one of embodiments 36-42, wherein the proteotypic peptide occurs in the Fc region of antibodies specific to the SARS-COV-2 antigen.

Embodiment 43 is the method of any one of embodiments 36-42a, wherein the sample is a serum, plasma or another biological fluid sample, preferably serum.

Embodiment 43a is the method of embodiment 43, wherein the sample is a processed sample of the biological sample.

Embodiment 43b is the method of embodiment 43a, wherein the biological sample is centrifuged, frozen and/or thawed.

Embodiment 44 is the method of any one of embodiments 36-43b, wherein the step ii further comprises one or more steps of denaturing, reducing, alkylating and/or diluting the one or more protein complexes.

Embodiment 45 is the method of any one of embodiments 36-44, wherein the quantitative mass spectrometry is a tandem mass spectrometry.

Embodiment 45a is the method of any one of embodiments 36-45, wherein the quantitative mass spectrometry comprises multiple reaction monitoring (MRM).

Embodiment 45b is the method of any one of embodiments 36-45, wherein the quantitative mass spectrometry comprises selected reaction monitoring (SRM).

Embodiment 45c is the method of any one of embodiments 36-45, wherein the quantitative mass spectrometry comprises a parallel reaction monitoring (PRM).

Embodiment 45d is the method of embodiment 45c, wherein the PRM comprises a C18 nano-LC connected to an ESI-Q-Orbitrap mass spectrometer.

Embodiment 46 is the method of any one of embodiments 36-45d, further comprising measuring the mass response of the intact mass of each of the proteotypic peptides.

Embodiment 46a is the method of embodiment 46, further comprising comparing the mass response of the intact mass of each of the proteotypic peptides to the reference curve of the corresponding proteotypic peptide.

Embodiment 46b is the method of any one of embodiments 36-45d, further comprising measuring the mass response of the fragment mass of each of the proteotypic peptides.

Embodiment 46c is the method of embodiment 46b, further comprising comparing the mass response of the fragment mass of each of the proteotypic peptides to the reference curve of the corresponding proteotypic peptide.

Embodiment 47 is the method of any one of embodiments 36-46c, wherein the absolute amount of the antibodies in the sample is expressed in weight concentration or molar concentration.

### EXAMPLES

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present application as defined by the present description.

### Example 1. Use of Proteotypic Peptide for the Determination of the Absolute Amount of RSV Antigen Specific IgG

This example describes the use of a proteotypic peptide, specific for any human IgG 1, 3 and 4, for the determination of the absolute amount of antigen specific IgG antibodies in anti-RSV-B F antigen (SEQ ID NO.11 for the RSV-B F antigen) IgG positive human serum by combining quantitative mass spectrometry (qMS) analysis and ELISA.

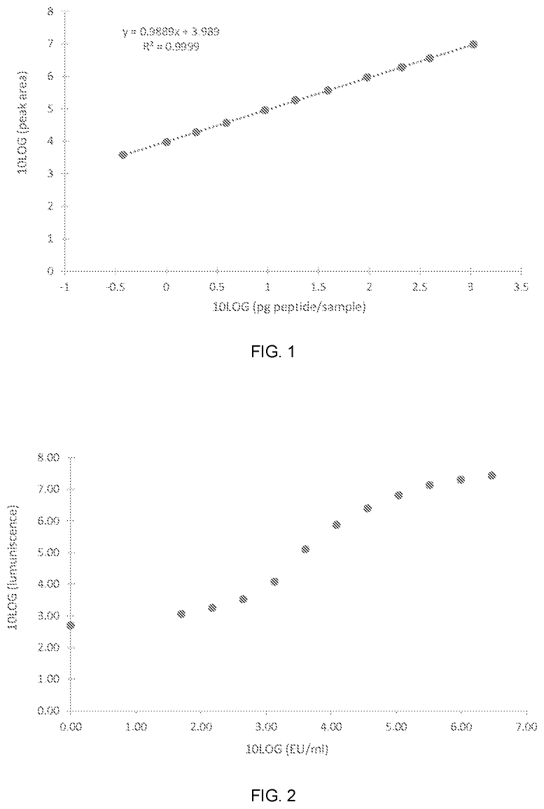

First step in the qMS analysis of the ELISA reference material (pooled IgG-containing serum isolated from viral infected individuals) is to select a proteotypic peptide, a peptide that only occurs in the specific protein of interest (i.e. human IgG1, 3 and 4 antibodies). For the proof of principle of the present invention, a peptide specific for the Fc region of human IgG 1, 3 and 4 was selected, namely VVSVLTVLHQDWLNGK (SEQ ID NO.1; Furlong, Ouyang et al. 2012; Furlong, Zhao et al. 2013). A synthetic version of this specific peptide was obtained and used to test its suitability, by creating a calibration curve of the proteotypic peptide, by adding known concentrations of the proteotypic peptide (“spiking”) in the range between 0.4 to 1000 pg/sample. The calibration samples were analyzed by reversed phase liquid chromatography, connected to an ESI-triple quad mass spectrometer in multiple reaction monitoring mode (MRM). In FIG. 1, linearity can be observed between the mass spectrometric MS2 response of the peptide (fragment mass of the peptide reported in Log 10 transformed peak area) and the known concentration of added peptide (reported in Log 10 transformed pg/sample) thereby making it suitable to use for quantitatively determining the amount of peptide in a mixture and subsequently of IgG. Hence, this linear dose response establishes that the qMS analysis is suitable for quantitatively determining the amount of human anti-RSV-B F protein antibodies in a sample mixture.

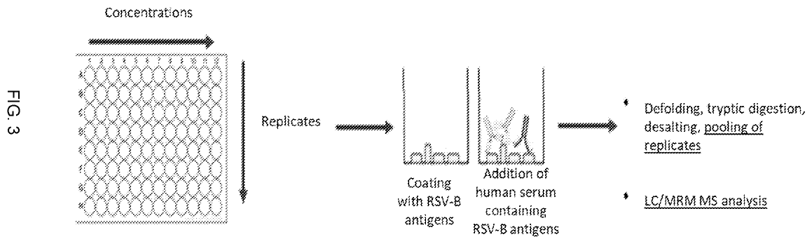

When performing an RSV-B F ELISA in the ordinary way (as described above), a luminescence read-out for the detection of RSV-B F specific human serum antibodies (result) is used. This luminescence read-out is arbitrary and measured in Relative Light Units (RLU). To make the result semi-quantitative, the result of a sample is expressed as ratio of an anti-RSV-B F protein IgG positive human serum reference expressed in EU/ml. The dilution curve obtained from the luminescence read-outs, measuring the antibody binding of the RSV-B F protein positive human serum reference in the ELISA is shown in FIG. 2. The linear part of this curve (±1350 to 328050 EU/ml) is used to quantify the immune response in unknown human serum samples. However, the quantity is then expressed in ELISA Units per ml (EU/ml) which is an arbitrary value. To make this value absolute, the curve needs to be measured by mass spectrometry.

To convert the arbitrary values in EU/mL into absolute values in pg IgG/ml, the reference standard curve samples of the ELISA reference standard curve were measured by mass spectrometry. This was achieved by performing the first two steps of the ordinary ELISA: (1) RSV-B F protein (SEQ ID NO.11) was coated to an ELISA plate, and (2) the reference standard curve samples, generated with the anti-RSV-B F protein antibody positive human serum, were added to the ELISA plate to obtain different concentrations (see FIG. 3). Afterwards, all non-bound antibodies were washed away. The remaining antibodies and proteins, bound to the plate, were removed and denatured by addition of 8 M Urea. Denatured antibodies and proteins from multiple wells were pooled (n=16 wells of a 96-well plate), based on the concentration of RSV-B F antigen positive serum, to increase the sensitivity during the analysis. After reduction and alkylation of the antibodies and proteins, the antibodies and proteins were diluted in 2 M urea, and trypsin was added for a 3 hour digestion at 37° C. Finally, the samples were desalted by solid phase extraction and analyzed by LC ESI triplequad analysis in MRM mode.

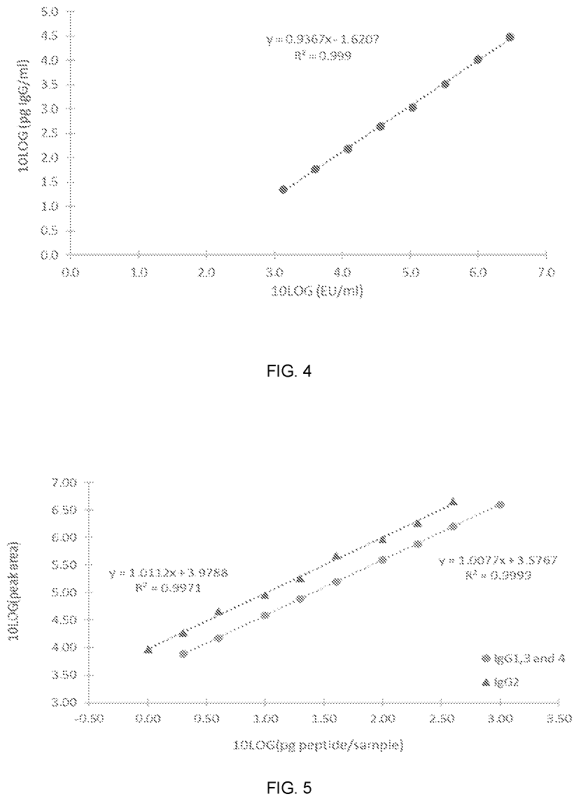

The obtained MS 2 peak areas for the different samples from the linear part of the ELISA reference standard curve were converted into Log10 transformed pg IgG/ml, using the equation of the linear regression from the curve shown in FIG. 1, multiplied by the serum dilution factor in the ELISA and subsequently plotted against the Log 10 transformed dilution of reference material in EU/mL (see Table 2 and FIG. 4). The results show that the mass spectrometric results are also linear in this part of the dilution curve. However, instead of obtaining spectrophotometric read-outs expressed in the arbitrary relative luminescence units (RLUs) on the Y-axis of the plot, it now shows an absolute amount of antigen binding IgG, expressed in pg IgG/ml.

TABLE 2
EU/ml, MS2 peak area and pg IgG/ml for linear part of ELISA dilution
curve based on quantitative mass spectrometric measurements

| | MS2 peak area | pg IgG/ | |
|---|---|---|---|
| EU/mL | (pg peptide/sample) | per well | pg IgG/ml |
| 2952450 | 584.777 | 3.1807 | 30320.5 |
| 984150 | 199.401 | 2.7134 | 10338.9 |
| 328050 | 62.788 | 2.2116 | 3255.5 |
| 109350 | 21.238 | 1.7408 | 1101.2 |
| 36450 | 8.674 | 1.3519 | 449.7 |
| 12150 | 2.956 | 0.8844 | 153.3 |
| 4050 | 1.145 | 0.4725 | 59.4 |
| 1350 | 0.426 | 0.0426 | 22.1 |

From this point on, the obtained formula in FIG. 4 (10 Log pg IgG/ml=(0.9367 * 10 Log EU/ml)−1.6207) can be applied to convert ELISA read-outs (expressed in EU/ml) from a regular ELISA to absolute quantities of IgG, expressed in pg IgG/ml. This will allow for expressing the quantities of IgG antibodies obtained in future luminescence ELISA dilution curves, in absolute quantities, expressed in pg IgG/ml. Although the ELISA read-out values may be experiment dependent, the absolute IgG amounts are valid for all experiments run with identical set up and materials or materials that have been shown to be analytically equivalent through bridging. Subsequently, all unknown serum samples can now be analyzed by the standard ELISA and the read-out (for example luminescence value) can be expressed relative to the reference material as ordinarily done, however the units of the IgG concentration will now be absolute values, expressed in pg IgG/ml.

### Example 2. Use of Proteotypic Peptide for the Determination of the Absolute Amount of HIV Antigen Specific IgG

This example describes the use of a proteotypic peptide, specific for any human IgG 1, 3 and 4 or IgG 2, for the determination of the absolute amount of antigen specific IgG antibodies in anti-HIV antigen (Mosaic gp140) IgG positive human serum by combining quantitative mass spectrometry (qMS) analysis and ELISA.

For this example, two proteotypic peptides were selected: First, the peptide that was also described in example 1, VVSVLTVLHQDWLNGK (SEQ ID NO. 1) that is specific for human IgG1, 3 and 4 antibodies; Second, VVSVLTVVHQDWLNGK (SEQ ID NO.2; Furlong, Ouyang et al. 2012; Furlong, Zhao et al. 2013) that is specific for human IgG2. Synthetic versions of these specific peptides were obtained and used to test their suitability, by creating a calibration curve of each of the proteotypic peptides, by adding known concentrations of the proteotypic peptide (“spiking”) in the range between 1 to 1000 pg/analyzed sample. The calibration samples were analyzed by reversed phase liquid chromatography, connected to an ESI-triple quad mass spectrometer in multiple reaction monitoring mode (MRM). In FIG. 5, linearity can be observed between the mass spectrometric MS2 response of both the peptides (fragment mass of the peptide reported in Log 10 transformed peak area) and the known concentration of added peptide (peptide reported in Log 10 transformed pg/sample), thereby making it suitable to use for quantitatively determining the amount of peptide in a mixture and subsequently of IgG. Hence, this linear dose response establishes that the qMS analysis is suitable for quantitatively determining the amount of human anti-HIV IgG antibodies in a sample mixture.

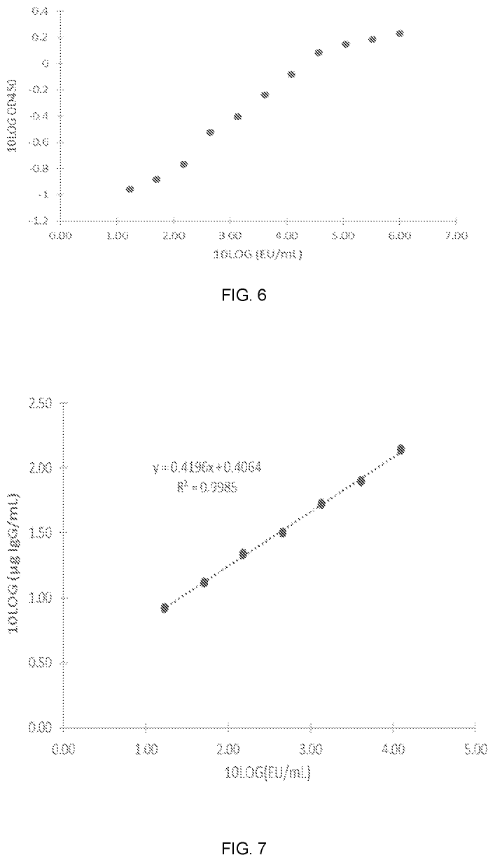

When performing this HIV ELISA in the ordinary way (as described above), an optical density at 450 nm (OD450) read-out for the detection of HIV specific human serum antibodies (result) is used. This OD450 read out is arbitrary and measured in Absorption units (AU). To make the result semi-quantitative, the result of a sample is expressed as ratio of an anti-HIV protein IgG positive human serum reference expressed in EU/ml. The dilution curve obtained from the OD450 read-outs, measuring the antibody binding of the HIV protein positive human serum reference in the ELISA is shown in FIG. 6. The linear part of this curve (±150 to 37000 EU/mL) is used to quantify the immune response in unknown human serum samples. However, the quantity is then expressed in ELISA Units per mL (EU/mL) which is an arbitrary value. To make this value absolute, the curve needs to be measured by mass spectrometry.

To convert the arbitrary values in EU/mL into absolute values in μg IgG/mL, the reference standard curve samples of the ELISA reference standard curve were measured by mass spectrometry. This was achieved by performing the first two steps of the ordinary ELISA: (1) Mosaic gp140 antigen was coated to an ELISA plate, and (2) the reference standard curve samples, generated with the anti-HIV protein/antigen antibody positive human serum, were added to the ELISA plate to obtain different concentrations (see FIG. 3). Afterwards, all non-bound antibodies were washed away. The remaining antibodies and proteins, bound to the plate, were removed and denatured by addition of 8 M Urea. Denatured antibodies and proteins from multiple wells were pooled (n=16 wells of a 96-well plate), based on the concentration of HIV antigen positive serum, to increase the sensitivity during the analysis. After reduction and alkylation of the antibodies and proteins, the antibodies and proteins were diluted in 2 M urea, and trypsin was added for a 3-hour digestion at 37° C. Finally, the samples were desalted by solid phase extraction and analyzed by LC ESI triplequad analysis in MRM mode.

The obtained MS 2 peak areas for the different samples from the linear part of the ELISA reference standard curve were converted into absolute quantities of IgG1, 3, and 4 and IgG2 respectively μg IgG/mL for both selected peptides, using the equations of the linear regression from the curve of IgG1, 3,and 4 and IgG2 as shown in FIG. 5. Subsequently the IgG1, 3, and 4 and IgG2 values were summated to obtain a total IgG concentration for each ELISA reference standard curve point. Finally, the obtained total IgG values were Log 10 transformed, and plotted against the Log 10 transformed dilution of reference material in EU/mL, multiplied by the serum dilution factor in the ELISA (see Table 3 and FIG. 7). The results show that the mass spectrometric results are also linear in this part of the dilution curve. However, instead of obtaining spectrophotometric read-outs expressed in the arbitrary relative OD450 units (AU) on the Y-axis of the plot, it now shows an absolute amount of antigen binding IgG, expressed in μg IgG/mL.

TABLE 3
MS2 peak, EU/mL and μg IgG/mL for linear part of ELISA dilution
curve based on quantitative mass spectrometric measurements

| | MS peak | | | | |
|---|---|---|---|---|---|
| | area IgG1, | MS peak | | | |
| | 3, and 4 | area IgG2 | IgG1, 3 | | |
| | (pg peptide/ | (pg peptide/ | and 4 | IgG 2 | Total IgG |
| EU/mL | sample) | sample) | μg/mL | μg/mL | μg/mL |
| 457 | 15.031 | 5.0737 | 6.235 | 2.121 | 8.356 |
| 1372 | 24.192 | 7.6283 | 10.035 | 3.189 | 13.224 |
| 4115 | 42.696 | 10.4008 | 17.711 | 4.348 | 22.059 |
| 12346 | 59.767 | 16.3340 | 24.792 | 6.828 | 31.620 |
| 37037 | 113.356 | 14.9715 | 47.020 | 6.259 | 53.279 |
| 111111 | 178.707 | 13.9813 | 74.128 | 5.845 | 79.973 |
| 333333 | 316.619 | 17.4588 | 131.334 | 7.299 | 138.633 |

From this point on, the obtained formula in FIG. 7 (10 Log μg IgG/mL=(0.420*10 Log EU/mL)+0.406) can be applied to convert ELISA read-outs (expressed in EU/mL) from a regular ELISA to absolute quantities of IgG, expressed in μg IgG/mL. This will allow for expressing the quantities of IgG antibodies obtained in future optical density ELISA dilution curves, in absolute quantities, expressed in μg IgG/mL. Although the ELISA read-out values may be experiment dependent, the absolute IgG amounts are valid for all experiments run with identical set up and materials or materials that have been shown to be analytically equivalent through bridging. Subsequently, all unknown serum samples can now be analyzed by the standard ELISA and the read-out (for example luminescence value) can be expressed relative to the reference material as ordinarily done, however the units of the IgG concentration will now be absolute values, expressed in μg IgG/mL.

### Example 3. Use of Proteotypic Peptide for the Determination of the Absolute Amount of SARS-COV-2 Antigen Specific IgG

This example describes the use of a proteotypic peptide, specific for any human IgG 1, 3 and 4 or IgG2, for the determination of the absolute amount of antigen specific IgG antibodies in SARS-COV-2 positive human serum by combining quantitative mass spectrometry (qMS) analysis and ELISA.

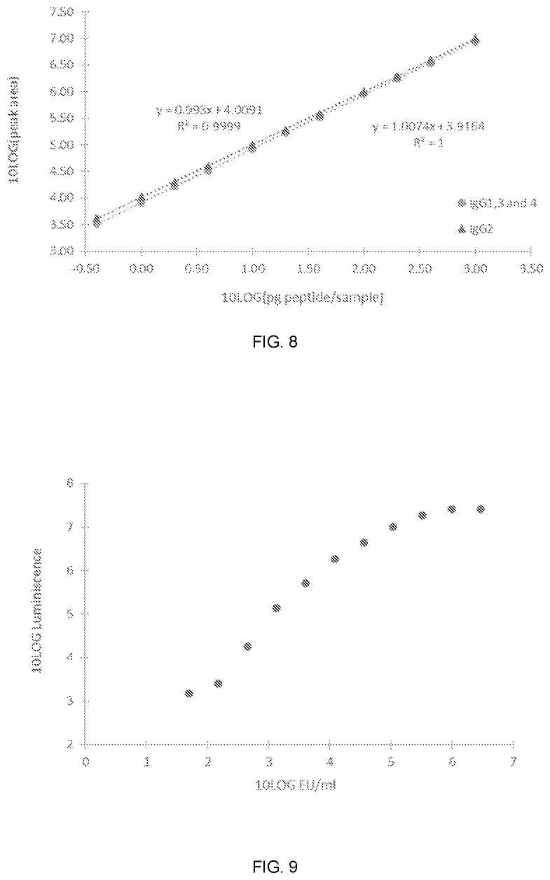

For this example, two proteotypic peptides were selected: First, the peptide that was also described in example 1, VVSVLTVLHQDWLNGK (SEQ ID NO. 1) that is specific for human IgG1, 3 and 4 antibodies. Second, VVSVLTVVHQDWLNGK (SEQ ID NO.2; Furlong, Ouyang et al. 2012; Furlong, Zhao et al. 2013) that is specific for human IgG2. Synthetic versions of these specific peptides were obtained and used to test their suitability, by creating a calibration curve of each of the proteotypic peptides, by adding known concentrations of the proteotypic peptide (“spiking”) in the range between 1 to 1000 pg/analyzed sample. The calibration samples were analyzed by reversed phase liquid chromatography, connected to an ESI-triple quad mass spectrometer in multiple reaction monitoring mode (MRM). In FIG. 8, linearity can be observed between the mass spectrometric MS2 response of both the peptides (fragment mass of the peptide reported in Log 10 transformed peak area) and the known concentration of added peptide (peptide reported in Log 10 transformed pg/sample), thereby making it suitable to use for quantitatively determining the amount of peptide in a mixture and subsequently of IgG. Hence, this linear dose response establishes that the qMS analysis is suitable for quantitatively determining the amount of human anti-SARS-COV-2 IgG antibodies in a sample mixture.

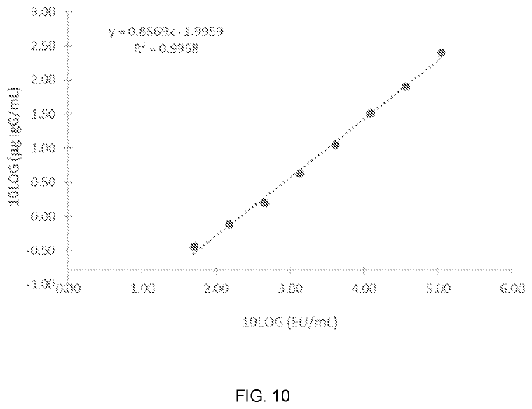

When performing this SARS-COV-2 ELISA in the ordinary way (as described above), a luminescence read-out for the detection of SARS-COV-2 specific human serum antibodies (result) is used. This luminescence read out is arbitrary and measured in Relative Light Units (RLU). To make the result semi-quantitative, the result of a sample is expressed as ratio of an anti-SARS-COV-2 protein IgG positive human serum reference expressed in EU/ml. The dilution curve obtained from the luminescence read-outs, measuring the antibody binding of the SARS-COV-2 protein positive human serum reference in the ELISA is shown in FIG. 9. The linear part of this curve (±450 to 33000 EU/mL) is used to quantify the immune response in unknown human serum samples. However, the quantity is then expressed in ELISA Units per mL (EU/mL) which is an arbitrary value. To make this value absolute, the curve needs to be measured by mass spectrometry.

To convert the arbitrary values in EU/mL into absolute values in μg IgG/mL, the reference standard curve samples of the ELISA reference standard curve were measured by mass spectrometry. This was achieved by performing the first two steps of the ordinary ELISA: (1) SARS-COV-2 antigen was coated to an ELISA plate, and (2) the reference standard curve samples, generated with the anti-SARS-COV-2 protein/antigen antibody positive human serum, were added to the ELISA plate to obtain different concentrations (see FIG. 3). Afterwards, all non-bound antibodies were washed away. The remaining antibodies and proteins, bound to the plate, were removed and denatured by addition of 8 M Urea. Denatured antibodies and proteins from multiple wells were pooled (n=16 wells of a 96-well plate), based on the concentration of SARS-COV-2 antigen positive serum, to increase the sensitivity during the analysis. After reduction and alkylation of the antibodies and proteins, the antibodies and proteins were diluted in 2 M urea, and trypsin was added for a 3-hour digestion at 37° C. Finally, the samples were desalted by solid phase extraction and analyzed by LC ESI triplequad analysis in MRM mode.

The obtained MS 2 peak areas for the different samples from the linear part of the ELISA reference standard curve were converted into absolute quantities of IgG1, 3, and 4 and IgG2 respectively μg IgG/mL for both selected peptides, using the equations of the linear regression for IgG1, 3, and 4 and IgG2 from the curve shown in FIG. 8. Subsequently the IgG1, 3, and 4 and IgG2 values were summated to obtain a total IgG concentration for each ELISA reference standard curve point. Finally, the obtained total IgG values were Log10 transformed, and plotted against the Log 10 transformed dilution of reference material in EU/mL, multiplied by the serum dilution factor in the ELISA (see Table 4 and FIG. 10). The results show that the mass spectrometric results are also linear in this part of the dilution curve. However, instead of obtaining spectrophotometric read-outs expressed in the arbitrary relative luminescence on the Y-axis of the plot, it now shows an absolute amount of antigen binding IgG, expressed in μg IgG/mL.

TABLE 4
MS2 peak, EU/mL and μg IgG/mL for linear part of ELISA dilution
curve based on quantitative mass spectrometric measurements

| | MS peak | | | | |
|---|---|---|---|---|---|
| | area IgG1, | MS peak | | | |
| | 3, and 4 | area IgG2 | IgG1, 3 | | |
| | (pg peptide/ | (pg peptide/ | and 4 | IgG 2 | Total IgG |
| EU/mL | sample) | sample) | μg/mL | μg/mL | μg/mL |
| 1350 | 0.28 | 11.41 | 0.228 | 0.138 | 0.366 |
| 4050 | 0.70 | 29.20 | 0.584 | 0.171 | 0.755 |
| 12150 | 1.74 | 71.97 | 1.439 | 0.141 | 1.580 |
| 36450 | 4.75 | 196.82 | 3.936 | 0.256 | 4.193 |
| 109350 | 12.95 | 537.17 | 10.743 | 0.502 | 11.246 |
| 328050 | 38.00 | 1576.25 | 31.525 | 1.129 | 32.654 |
| 984150 | 93.25 | 3868.03 | 77.361 | 2.274 | 79.635 |
| 2952450 | 294.00 | 12195.19 | 243.904 | 7.048 | 250.952 |

From this point on, the obtained formula in FIG. 10 (10 Log μg IgG/mL=(0.857*10 Log EU/mL)−2.00) can be applied to convert ELISA read-outs (expressed in EU/mL) from a regular ELISA to absolute quantities of IgG, expressed in μg IgG/mL. This will allow for expressing the quantities of IgG antibodies obtained in future optical density ELISA dilution curves, in absolute quantities, expressed in μg IgG/mL. Although the ELISA read-out values may be experiment dependent, the absolute IgG amounts are valid for all experiments run with identical set up and materials or materials that have been shown to be analytically equivalent through bridging. Subsequently, all unknown serum samples can now be analyzed by the standard ELISA and the read-out (for example luminescence value) can be expressed relative to the reference material as ordinarily done, however the units of the IgG concentration will now be absolute values, expressed in μg IgG/mL.

It is understood that the examples and embodiments described herein are for illustrative purposes only, and that changes could be made to the embodiments described above without departing from the broad inventive concept thereof. It is understood, therefore, that this invention is not limited to the particular embodiments disclosed, but it is intended to cover modifications within the spirit and scope of the invention as defined by the appended claims.

### REFERENCES

1. Aydin, S. (2015). “A short history, principles, and types of ELISA, and our laboratory experience with peptide/protein analyses using ELISA.” Peptides 72: 4-15.
2. Calderon-Celis, F., J. R. Encinar and A. Sanz-Medel (2018). “Standardization approaches in absolute quantitative proteomics with mass spectrometry.” Mass Spectrometry Reviews 37(6): 715-737.
3. Furlong, M. T., Z. Ouyang, S. Wu, J. Tamura, T. Olah, A. Tymiak and M. Jemal (2012). “A universal surrogate peptide to enable LC-MS/MS bioanalysis of a diversity of human monoclonal antibody and human Fc-fusion protein drug candidates in pre-clinical animal studies.” Biomedical Chromatography 26(8): 1024-1032.
4. Furlong, M. T., S. Zhao, W. Mylott, R. Jenkins, M. Gao, V. Hegde, J. Tamura, A. Tymiak and M. Jemal (2013). “Dual universal peptide approach to bioanalysis of human monoclonal antibody protein drug candidates in animal studies.” Bioanalysis 5(11): 1363-1376.
5. Halquist MS and Thomas Karnes H (2011). “Quantitative liquid chromatography tandem mass spectrometry analysis of macromolecules using signature peptides in biological fluids.” Biomedical Chromatography; 25: 47-58.
6. Pan, S., R. Aebersold, R. Chen, J. Rush, D. R. Goodlett, M. W. McIntosh, J. Zhang and T. A. Brentnall (2009). “Mass Spectrometry Based Targeted Protein Quantification: Methods and Applications.” Journal of Proteome Research 8(2): 787-797.

1. A method of determining an absolute amount of antibodies specific to an antigen in a sample, the method comprising:
i. contacting a calibration material containing the antibodies with the antigen to form one or more protein complexes comprising the antigen and the antibodies bound to the antigen;
ii. determining the relative amount of the antibodies in the calibration material by an ELISA detection method;
iii. repeating step i by contacting the calibration material with the antigen to form one or more protein complexes comprising the antigen and the antibodies bound to the antigen;
iv. isolating the one or more protein complexes;
v. digesting the one or more protein complexes to generate a mixture comprising a proteotypic peptide of the antibodies, wherein the proteotypic peptide occur in the Fc region of the antibodies;
vi. subjecting the mixture to quantitative mass spectrometry to measure a mass response of the proteotypic peptide;
vii. determining the absolute amount of the antibodies in the calibration material by comparing the mass response of the proteotypic peptide to a reference curve of the proteotypic peptide, wherein the reference curve relates a mass response of the proteotypic peptide to an absolute amount of the proteotypic peptide;
viii. establishing a conversion factor to correlate the relative amount of the antibodies in the calibration material as determined by the ELISA in step ii to the absolute amount of the antibodies in the calibration material as determined in step vii;
ix. contacting the sample with the antigen to form one or more protein complexes comprising the antigen and the antibodies bound to the antigen;
x. determining the relative amount of the antibodies in the sample by the ELISA detection method; and
xi. applying the conversion factor to determine the absolute amount of the antibodies in the sample.

2. The method of claim 1, further comprising obtaining the reference curve of the proteotypic peptide before step i.

3. The method of claim 1, wherein in step vii, an absolute amount of the proteotypic peptide is first determined by comparing the mass response of the proteotypic peptide to the reference curve of the proteotypic peptide, and then the absolute amount of the antibodies in the calibration material is determined based on the absolute amount of the proteotypic peptide.

4. The method of claim 1, wherein the sample is a sample obtained from a subject administered with a vaccine comprising the antigen or a related antigen from the same virus, or from a subject who has been in contact with a virus comprising the antigen.

5. The method of claim 4, wherein the vaccine is a preventive vaccine or therapeutic vaccine.

6. The method of claim 1, wherein the antigen is an RSV-B F antigen.

7. The method of claim 1, wherein the antibodies in the sample bind to the same or different epitopes of the antigen.

8. The method of claim 1, wherein the antibodies comprise one or more of immunoglobulin G (IgG).

9. The method of claim 1, wherein the sample is a serum, plasma or another biological fluid sample.

10. The method of claim 1, wherein the calibration material is a biological fluid sample.

11. The method of claim 1, wherein the step v further comprises one or more steps of denaturing, reducing, alkylating and/or diluting the one or more protein complexes.

12. The method of claim 1, wherein the antibodies comprise one or more of IgG1, IgG2, IgG3 and IgG4.

13. The method of claim 1, wherein the proteotypic peptide comprises the amino acid sequence of SEQ ID NO. 1 or SEQ ID NO. 2.

14. The method of claim 1, further comprising subjecting the mixture to liquid chromatography (LC) prior to step vi.

15. The method of claim 1, wherein the quantitative mass spectrometry is a tandem mass spectrometry, comprises multiple reaction monitoring (MRM) or selected reaction monitoring (SRM), and/or.

16. (canceled)

17. (canceled)

18. The method of claim 1, further comprising measuring the mass response of the intact mass of the proteotypic peptide comprising comparing the mass response of the intact mass of the prototypic peptide to the reference curve of the proteotypic peptide.

19. (canceled)

20. A method of determining an absolute amount of antibodies specific to an RSV antigen in a sample from a subject administered with a vaccine comprising the RSV antigen, the method comprising:
i. contacting the sample with the RSV antigen to form one or more protein complexes comprising the RSV antigen and the antibodies bound to the antigen;
ii. isolating the one or more protein complexes;
iii. digesting the one or more protein complexes to generate a first mixture comprising a proteotypic peptide having the amino acid sequence of SEQ ID NO. 1;
iv. subjecting the first mixture to liquid chromatography (LC) to generate a second mixture;
v. subjecting the second mixture to a quantitative mass spectrometry to thereby generate a mass spectrum of the proteotypic peptide;
vi. measuring a mass response of the proteotypic peptide from the mass spectrum; and
vii. determining the absolute amount of the antibodies specific to the RSV antigen in the sample by comparing the mass response of the proteotypic peptide to a reference curve of the proteotypic peptide, wherein the reference curve relates a mass spectrometric response of the proteotypic peptide to an absolute amount of the proteotypic peptide.

21. The method of claim 20, further comprising obtaining the reference curve of the proteotypic peptide before step i.

22. The method of claim 21, wherein in step vii, an absolute amount of the proteotypic peptide is first determined by comparing the mass response of the proteotypic peptide to the reference curve of the proteotypic peptide, and then the absolute amount of the antibodies specific to the RSV antigen is determined based on the absolute amount of the proteotypic peptide.

23. The method of claim 21, wherein the quantitative mass spectrometry is a tandem mass spectrometry and/or comprises multiple reaction monitoring (MRM), selected reaction monitoring (SRM), or a parallel reaction monitoring (PRM).

24. (canceled)

25. The method of claim 20, further comprising measuring the mass response of the intact mass of the proteotypic peptide.

26. The method of claim 1, further comprising comparing the mass response of the intact mass of the proteotypic peptide to the reference curve of the proteotypic peptide.

27. A method of determining an absolute amount of antibodies specific to an HIV antigen in a sample from a subject administered with a vaccine comprising the HIV antigen, the method comprising:
i. contacting the sample with the HIV antigen to form one or more protein complexes comprising the HIV antigen and the antibodies bound to the antigen;
ii. isolating the one or more protein complexes;
iii. digesting the one or more protein complexes to generate a first mixture comprising a proteotypic peptide having the amino acid sequence of SEQ ID NO. 1 and a proteotypic peptide having the amino acid sequence of SEQ ID NO. 2;
iv. subjecting the first mixture to liquid chromatography (LC) to generate a second mixture;
v. subjecting the second mixture to a quantitative mass spectrometry to thereby generate a mass spectrum of each of the proteotypic peptides;
vi. measuring a mass response of each of the proteotypic peptides from the mass spectrum; and
vii. determining the absolute amount of the antibodies specific to the HIV antigen in the sample by comparing the mass response of each of the proteotypic peptides to a reference curve of each of the proteotypic peptides, wherein the reference curve relates a mass spectrometric response of the proteotypic peptide to an absolute amount of the proteotypic peptide.

28-35. (canceled)

36. A method of determining an absolute amount of antibodies specific to a SARS-COV-2 antigen in a sample from a subject administered with a vaccine comprising the SARS-COV-2 antigen, the method comprising:
i. contacting the sample with the SARS-COV-2 antigen to form one or more protein complexes comprising the SARS-COV-2 antigen and the antibodies bound to the antigen;
ii. isolating the one or more protein complexes;
iii. digesting the one or more protein complexes to generate a first mixture comprising a proteotypic peptide having the amino acid sequence of SEQ ID NO. 1 and a proteotypic peptide having the amino acid sequence of SEQ ID NO. 2;
iv. subjecting the first mixture to liquid chromatography (LC) to generate a second mixture;
v. subjecting the second mixture to a quantitative mass spectrometry to thereby generate a mass spectrum of each of the proteotypic peptides;
vi. measuring a mass response of each of the proteotypic peptides from the mass spectrum; and
vii. determining the absolute amount of the antibodies specific to the SARS-COV-2 antigen in the sample by comparing the mass response of each of the proteotypic peptides to a reference curve of each of the proteotypic peptides, wherein the reference curve relates a mass spectrometric response of the proteotypic peptide to an absolute amount of the proteotypic peptide.

37-44. (canceled)


SEQUENCE LISTING (11 sequences)

SEQ ID NO: 1 - protein, 16 aa, Homo sapiens
VVSVLTVLHQDWLNGK

SEQ ID NO: 2 - protein, 16 aa, Homo sapiens
VVSVLTVVHQDWLNGK

SEQ ID NO: 3 - protein, 7 aa, Homo sapiens
DTLMISR

SEQ ID NO: 4 - protein, 8 aa, Homo sapiens
ALPAPIEK

SEQ ID NO: 5 - protein, 8 aa, Homo sapiens
GLPAPIEK

SEQ ID NO: 6 - protein, 8 aa, Homo sapiens
GLPSSIEK

SEQ ID NO: 7 - protein, 22 aa, Homo sapiens
GFYPSDIAVEWESNGQPENNYK

SEQ ID NO: 8 - protein, 22 aa, Homo sapiens
GFYPSDISVEWESNGQPENNYK

SEQ ID NO: 9 - protein, 22 aa, Homo sapiens
GFYPSDIAVEWESSGQPENNYN

SEQ ID NO: 10 - protein, 9 aa, Homo sapiens
DTLMISRPH

SEQ ID NO: 11 - protein, 558 aa, Homo sapiens
MELLILKANAITTILTAVTFCFASGQNITEEFYQSTCSAVSRGYLSALRTGWYTSVITIELSNIKETKCNGTDTKVKLIKQELDKYKNAVTELQLLMQNTQAANNRARREAPQYMNYTINTTKNLNVSISKKRKRRFLGFLLGVGSAIASGMAVSKVLHLEGEVNKIKNALQLTNKAVVSLSNGVSVLTSRVLDLKNYINNQILPMVNRQSCRIPNIETVIEFQQKNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSNEFYASISQVNEKINQSLAFIRRSDELLSAIGGYIPEAPRDGQAYVRKDGEWVLLSTFLLPATGGSGGSEPEA